SEQUENCE LISTING (2 sequences)

SEQ ID NO: 1 - protein, 37 aa, Artificial sequence
  Synthesized
YTSLIHSLIEESQNQQEKNEQELLELDKWASLWNWFC

SEQ ID NO: 2 - protein, 36 aa, Artificial sequence
  Synthesized
YTSLIHSLIEESQNQQEKNEQELLELDKWASLWNWF

(19) United States
(12) Patent Application Publication
(10) Pub. No.: US 2008/0213249 A1
(43) Pub. Date: Sep. 4, 2008

(54) Use of Retro-Aldol Reaction to Generate Reactive Vinyl Ketone for Attachment to Antibody Molecules by Michael Addition Reaction for Use in Immunostaining and Immunotargeting

(75) Inventors: Subhash C. Sinha, San Diego, CA (US); Carlos F. Barbas, Solana Beach, CA (US); Richard A. Lerner, San Diego, CA (US)

Correspondence Address: CATALYST LAW GROUP, APC, 9710 SCRANTON ROAD, SUITE S-170, SAN DIEGO, CA, 92121, US

(73) Assignee: THE SCRIPPS RESEARCH INSITUTE, La Jolla, CA (US)

(21) Appl. No.: 11/774,149

(22) Filed: Jul. 6, 2007

Related U.S. Application Data
(60) Provisional application No. 60/819,234, filed on Jul. 7, 2006.

(51) Int. Cl. A61K 39/395; A61P 35/00; C07K 16/00; C07K 1/00; C07K 5/10

(52) U.S. Cl. 424/130.1; 530/387.1; 530/330; 530/300

(57) ABSTRACT

The present invention is directed to methods for formation of a chemically programmed antibody comprising the steps of: (1) reacting a conjugate comprising a signal module covalently linked to a proadapter with a catalytic moiety selected from the group consisting of a catalytic antibody and a Fab fragment of a catalytic antibody, wherein the proadapter includes therein a precursor to a reactive moiety activated to a reactive moiety by a reaction catalyzed by the catalytic moiety; and (2) crosslinking the reactive moiety to a side chain of an amino acid residue in the active site of the catalytic moiety to produce the chemically programmed antibody. The invention also encompasses chemically programmed antibodies formed by these methods, methods for their use, and pharmaceutical compositions, as well as proadaptors and conjugates including them. Chemically programmed antibodies are useful for the treatment of cancer, particularly in metastasis.

### CROSS-REFERENCE TO RELATED APPLICATIONS

This application claims priority from U.S. Provisional Application Ser. No. 60/819,234 by Barbas, III et al., entitled “Use of Retro-Aldol Reaction to Generate Reactive Vinyl Ketone for Attachment to Antibody Molecules by Michael Addition for Use in Immunostaining and Immunotargeting,” filed Jul. 7, 2006, which is incorporated herein in its entirety by this reference.

### STATEMENT REGARDING FEDERAL RESEARCH AND DEVELOPMENT

This invention was made with United States government support under the National Institutes of Health Grant No. 5R01CA104045 and under Department of Defense Grant W81XWH-04-1-0717. The United States government, therefore, has certain rights in this invention.

### BACKGROUND OF THE INVENTION

This invention is directed to methods of labeling the Fc portion of antibody molecules and related molecules including Fc regions for immunostaining and immunotargeting. In particular, this invention is directed to methods of using the retro aldol reaction to generate reactive vinyl ketone species for the labeling or targeting of antibodies and other protein molecules.

Antibodies are biological macromolecules with highly defined specificity. This specificity arises from the unique way the antibodies are generated. The use of antibody molecules in immunoassay, immunostaining, or immunotargeting encompasses a broad variety of applications, including in in vitro immunohistochemistry or immunocytochemistry and in in vivo labeling and detection.

Naturally-occurring immunoglobulins are tetramers with the general structure L₂H₂, with L being a so-called “light chain,” typically with a molecular weight of about 25,000 and H being a so-called “heavy chain,” typically with a molecular weight of 50,000. In naturally-occurring immunoglobulins, the two light chains and the two heavy chains are identical; these chains are held together by interchain disulfide bonds. Intrachain disulfide bonds also contribute to the stability of the antibody molecule. However, naturally-occurring antibodies are specific for one and only one antigen, the antigen binding to the complementarity-determining regions (CDRs) formed by the hypervariable regions of both the light chain and the heavy chain. This gives rise to the so-called “one antibody-one target” axiom. There is a great need for methods that can modify the specificity of antibodies so a single antibody, perhaps with particularly desirable properties, can be retargeted against antigens other than the antigen against which the antibody was originally formed. Such methods would allow the use of antibodies against multiple targets.

Immunoglobulins are divided into classes depending on the type of heavy chain found therein. The possible heavy chain molecules are designated γ, μ, α, ε, and δ, which give rise to immunoglobulins of class IgG, IgM, IgA, IgE, and IgD, respectively. Of these classes, the most common and the most frequently utilized is IgG. The discussion below therefore focuses on IgG immunoglobulins, with the understanding that it is also applicable to immunoglobulins of other classes unless excluded.

In immunoglobulins, such as IgG, there are regions or domains that provide specific functions. The presence of these domains is a consequence of the structure of the molecule. Both heavy chains and light chains include variable (V) regions and constant (C) regions. The antigen-binding site includes only a portion of the variable regions of both H and L chains, which include the actual amino acids responsible for the specific binding of the corresponding antigen by the antibody; these amino acids are referred to as the hypervariable region or the complementarity-determining regions (CDRs). The V regions include the amino-terminal portions of both H and L chains. The carboxyl-terminal portion of the H chains forms a region known as Fc. The Fc region plays no direct role in antigen binding, but is responsible for a number of effector functions, such as complement fixation and the generation of antibody-dependent cellular cytotoxicity (ADCC).

Therefore, there is a particular need for methods that can be used for modification of antibodies so that they can be retargeted, obviating the need to generate a new antibody against each new antigen. Preferably, such methods would retain the effector functions mediated by the Fc region of the antibody. Preferably, such retargeted or modified antibodies could be used in vivo for therapy such as for therapy of metastatic malignancies.

### SUMMARY OF THE INVENTION

One aspect of the present invention is a method for producing a chemically programmed antibody comprising the steps of:

(1) reacting a conjugate comprising a signal module covalently linked to a proadapter with a catalytic moiety selected from the group consisting of a catalytic antibody and a Fab fragment of a catalytic antibody, wherein the proadapter includes therein a precursor to a reactive moiety activated to a reactive moiety by a reaction catalyzed by the catalytic moiety; and

(2) crosslinking the reactive moiety to a side chain of an amino acid residue in the active site of the catalytic moiety to produce the chemically programmed antibody.

Typically, the reaction catalyzed by the catalytic moiety is a retro-aldol reaction, and the catalytic moiety has aldolase activity. Typically, the catalytic moiety is a catalytic antibody such as 38C2. Typically, the proadapter includes therein a tertiary aldol moiety, the tertiary aldol moiety is converted to a reactive vinyl ketone moiety by the retro-aldol reaction catalyzed by the catalytic moiety, and the vinyl ketone moiety reacts with a lysine residue in the active site of the catalytic moiety via Michael addition.

A preferred proadapter comprises a molecule selected from the group consisting of:

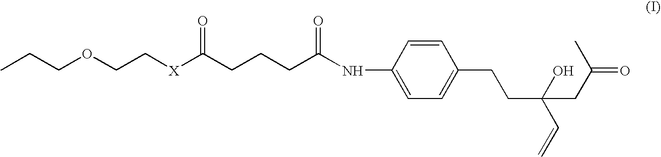

(1) a molecule of formula (1) wherein X is selected from the group consisting of NH and —CH₂NH and n is 2 or 3; and

(2) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule.

Particularly preferred proadapters are molecules of formula (I).

Alternatively, a proadapter suitable for use in methods and compositions according to the present invention comprises a molecule selected from the group consisting of:

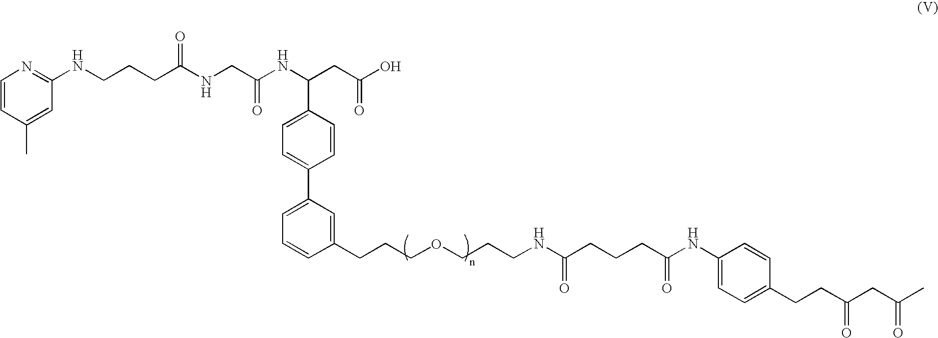

(a) a molecule of formula (V); and

(b) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule.

Typically, in this alternative, the proadapter is a molecule of formula (V).

A large variety of signal modules can be used. In one useful alternative, the signal module specifically targets an integrin, such as an integrin selected from the group consisting of αᵥβ₃, αᵥβ₅, and αᵥβ₆.

The method can be performed in vivo or in vitro.

Another aspect of the present invention is a chemically programmed antibody formed by the method of the invention as described above.

Yet another aspect of the present invention is a method of treatment of a disease or condition treatable by the administration of a chemically programmed antibody comprising the step of administering a therapeutically effective quantity of a chemically programmed antibody according to the present invention to treat the disease or condition. In particular, the condition can be cancer, such as metastatic breast cancer or metastatic melanoma.

An alternative treatment method relies on the in vivo generation of the chemically programmed antibody. In general, this method comprises:

(1) administering a therapeutically effective quantity of a catalytic moiety that can form a chemically programmed antibody according to the present invention when reacted with a proadaptor;

(2) administering a therapeutically effective quantity of a proadaptor that can form the chemically programmed antibody when reacted with the catalytic moiety; and

(3) reacting the catalytic moiety and proadaptor in vivo to form the chemically programmed antibody in a therapeutically effective quantity to treat the disease or condition.

As another alternative, a therapeutically effective quantity of a nucleic acid encoding a catalytic moiety that can form the chemically programmed antibody when reacted with a proadaptor can be administered instead of the catalytic moiety itself.

Another aspect of the present invention is a proadapter as described above.

Yet another aspect of the present invention is a conjugate comprising a proadapter as described above and a signal module covalently linked to the proadapter. A large variety of signal modules can be used.

### BRIEF DESCRIPTION OF THE DRAWINGS

The following invention will become better understood with reference to the specification, appended claims, and accompanying drawings, where:

FIG. 1 is a general schematic diagram showing the formation of cell-targeting antibody constructs based on adapter (A) and proadapter (B) approaches by using a β-diketone-equipped low-molecular weight targeting agent (“signal module”) and acetone adduct of the vinyl ketone-equipped targeting agent respectively (TA=targeting agent).

FIG. 2 shows structures of the αᵥβ₃ integrin-targeting antagonists equipped with an acetone adduct of a vinyl ketone, a vinyl ketone, or a diketone for chemical programming of the aldolase antibody.

FIG. 3 shows flow cytometry histograms showing the binding of 38C2-3b, 38C2-3c, and 38C2 alone (A) and binding of serial dilutions of 38C2-3b to MDA-MB-231 cells. In A, 38C2-3b and 38C2-3c prepared from 38C2 (1 eq) and 3a or 3c (2 eq) were diluted to 25 μg/ml. In B, the 38C2-3b (25 μg/ml) construct used in A was further diluted 5×, 25×, and 125×. In all experiments, 38C2 alone was used at 25 μg/ml, LM609 was used at 1:100 dilution, and FITC-conjugated goat anti-mouse secondary antibodies were used for detection. The y axis gives the number of events in linear scale, the x axis gives the fluorescence intensity in logarithmic scale.

FIG. 4 shows the results of a cell uptake assay using integrin αᵥβ₃-targeting 38C2 constructs (38C2-3b and 3802-3c) (A) and compounds 3a and 3c and mAb 38C2 in MDA-MB-231 cells (B). Antibody constructs 38C2-3b or 38C2-3c and antibody 38C2 alone were used at 5 μg/ml in PBS buffer. Compounds 3a and 3c were used at a 66.7 μM concentration (twice the concentration of 38C2-3b or 38C2-3c). FITC-conjugated goat anti-mouse secondary antibodies were used for detection.

FIG. 5 shows the effect of 38C2-3b on MDA-MB-231 pulmonary metastasis. SCID mice were injected intravenously with MDA-MB-231 cells pretreated with 50 μg of 38C2-3b, 0.3 μg of compound 1a, or 50 μg of 38C2, followed by additional treatments on days 2 and 4. Mice were killed on day 41, representative lungs from the treatment groups were harvested, and metastatic foci were counted in representative sections. Sections of lungs from treatment groups 38C2 (A), 1a (B), and 38C2-3b (C) are shown. The mean number of metastatic foci per group (n=5) with standard deviation are shown in D. ** statistical analysis by the Tukey-Kramer multiple comparison test demonstrated that the difference between the 38C23b-treated and the 38C2-treated group was significant (P<0.05). The Student t test also revealed significant differences between the 38C2-3b-treated and 38C2-treated group (P<0.01) and the 38C2-3b treated and 1a-treated group (P<0.05).

FIG. 6 (Scheme 1) depicts the synthesis of the αᵥβ₃ integrin-targeting agents; a, trifluoroacetic acid, CH₂Cl₂, anisole, then 6 or 7, Et₃N, and CH₃CN.

FIG. 7 (Scheme 2) depicts the synthesis of the integrin αᵥβ₆ targeting agents of Formula (IV) and (V) as described in Example 2.

### DETAILED DESCRIPTION OF THE INVENTION

### Definitions

As used herein, the term “nucleic acid,” “nucleic acid Sequence,” “polynucleotide,” or similar terms, refers to a deoxyribonucleotide or ribonucleotide oligonucleotide or polynucleotide, including single- or double-stranded forms, and coding or non-coding (e.g., “antisense”) forms. The term encompasses nucleic acids containing known analogues of natural nucleotides. The term also encompasses nucleic acids including modified or substituted bases as long as the modified or substituted bases interfere neither with the Watson-Crick binding of complementary nucleotides or with the binding of the nucleotide sequence by proteins that bind specifically, such as zinc finger proteins. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl)glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). Bases included in nucleic acids include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N⁶-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N⁶-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N⁶-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. DNA may be in the form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. Watson et al., Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) structure and/or activity, e.g., antibody activity, enzymatic activity, or receptor activity. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or H is; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations” when the three-dimensional structure and the function of the protein to be delivered are conserved by such a variation.

As used herein, the term “expression vector” refers to a plasmid, virus, phagemid, or other vehicle known in the art that has been manipulated by insertion or incorporation of heterologous DNA, such as nucleic acid encoding the fusion proteins herein or expression cassettes provided herein. Such expression vectors typically contain a promoter sequence for efficient transcription of the inserted nucleic acid in a cell. The expression vector typically contains an origin of replication, a promoter, as well as specific genes that permit phenotypic selection of transformed cells.

As used herein, the term “host cells” refers to cells in which a vector can be propagated and its DNA expressed. The term also includes any progeny of the subject host cell. It is understood that all progeny may not be identical to the parental cell since there may be mutations that occur during replication. Such progeny are included when the term “host cell” is used. Methods of stable transfer where the foreign DNA is continuously maintained in the host are known in the art.

As used herein, an expression or delivery vector refers to any plasmid or virus into which a foreign or heterologous DNA may be inserted for expression in a suitable host cell—i.e., the protein or polypeptide encoded by the DNA is synthesized in the host cells system. Vectors capable of directing the expression of DNA segments (genes) encoding one or more proteins are referred to herein as “expression vectors”. Also included are vectors that allow cloning of cDNA (complementary DNA) from mRNAs produced using reverse transcriptase.

As used herein, a gene refers to a nucleic acid molecule whose nucleotide sequence encodes an RNA or polypeptide. A gene can be either RNA or DNA, Genes may include regions preceding and following the coding region (leader and trailer) as well as intervening sequences (introns) between individual coding segments (exons).

As used herein, the term “isolated” with reference to a nucleic acid molecule or polypeptide or other biomolecule means that the nucleic acid or polypeptide has been separated from the genetic environment from which the polypeptide or nucleic acid were obtained. It may also mean that the biomolecule has been altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” is meant that the nucleic acid is free of the coding sequences of those genes that, in a naturally-occurring genome immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

“Isolated” or “purified” as those terms are used to refer to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the protein of interest can be present at various degrees of purity in these preparations. Particularly for proteins, the procedures may include for example, but are not limited to, ammonium sulfate fractionation, gel filtration, ion exchange change chromatography, affinity chromatography, density gradient centrifugation, electrofocusing, chromatofocusing, and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

One aspect of the invention is a method for producing a chemically programmed antibody comprising the steps of:

(1) reacting a conjugate comprising a signal module covalently linked to a proadapter with a catalytic moiety selected from the group consisting of a catalytic antibody and a Fab fragment of a catalytic antibody, wherein the proadapter includes therein a precursor to a reactive moiety activated to a reactive moiety by a reaction catalyzed by the catalytic moiety; and

(2) crosslinking the reactive moiety to a side chain of an amino acid residue in the active site of the catalytic moiety to produce the chemically programmed antibody.

The proadapter approach provides increased specificity of crosslinking to the catalytic moiety, as described in greater detail in Example 1. The requirement for activation of the precursor to the reactive moiety ensures that the reactive moiety does not react at undesired sites; instead, the reactive moiety reacts at a site within the active site of the catalytic moiety so that the reactive moiety is crosslinked to a side chain of an amino acid residue in the active site of the catalytic moiety. This produces a chemically programmed antibody. In effect, with the reactive moiety crosslinked to a side chain of an amino acid residue in the active site of the catalytic moiety, the antibody becomes a “shell” whose specificity is now controlled by the signal module. When a complete antibody molecule is used, the effector functions mediated by the Fc portion of the antibody molecule remain intact, and those effector functions are now targeted by the signal module. This means that the effective specificity of the antibody has been reprogrammed and the original specificity of the antibody has been abolished. This combines the merits of traditional small-molecule drug design with immunotherapy. It also allows breakage of the traditional “one antibody-one target” axiom by allowing the same antibody molecule to be used to target a large variety of possible targets, depending on the particular signal module used. This provides greatly improved utility for antibody molecules without the need of generating a new antibody molecule for each target.

Typically, the reaction catalyzed by the catalytic moiety is a retro-aldol reaction. When the reaction catalyzed by the catalytic moiety is a retro-aldol reaction, the catalytic moiety typically has aldolase activity. An activity of a catalytic moiety that has aldolase activity is the catalytic antibody 38C2, whose use is described in Example 1, below.

Although 38C2 is a preferred catalytic moiety that has aldolase activity, its use is not required; other catalytic moieties, such as catalytic antibodies or Fab fragments thereof that have aldolase activity, can be used, as well as muteins of these catalytic moieties. Typically, these muteins differ from their parent molecules by no more than two conservative amino acid substitutions while substantially retaining the catalytic activity of their parent molecules before the introduction of conservative amino acid substitutions. As used herein, the term “substantially retaining the catalytic activity” is defined as possessing a Vₘₐₓ for catalyzing the reaction on a preferred substrate of the parent molecule of no less than about 80% of the Vₘₐₓ of the parent molecule. In this context, the term “conservative amino acid substitution” is defined as one of the following substitutions: Ala/Gly or Ser; Arg/Lys; Asn/Gin or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. Preferably, the mutein differs from the parent molecule by no more than one conservative amino acid substitution. Methods for generating such muteins, such as site-directed mutagenesis techniques, are well known in the art.

Typically, the proadapter includes therein a tertiary aldol moiety. The tertiary aldol moiety can be converted to a reactive vinyl ketone moiety by the retro-aldol reaction catalyzed by the catalytic moiety. The vinyl ketone moiety then reacts with a lysine residue in the active site of the catalytic moiety via Michael addition. Further details on this sequence of reactions are provided in Examples 1 and 2.

In one alternative, the proadapter comprises a molecule selected from the group consisting of:

(a) a molecule of formula (I) wherein X is selected from the group consisting of NH and —CH₂NH and n is 2 or 3; and

(b) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule. As used herein, the term “lower alkyl” refers to an alkyl group having 1 to about 8 carbon atoms, more typically 1 to 6 carbon atoms. As used herein, the term “alkoxy” refers to an alkyl-O— group wherein alkyl is a lower alkyl group as previously described. As used herein, the term “halo” refers to fluoride, chloride, bromide, or iodide.

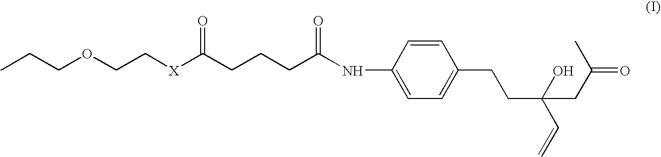

The proadapter is then converted into an adapter of formula (II) by the retro-aldol reaction to yield a vinyl ketone reactive with the lysine residue of the active site of the catalytic moiety

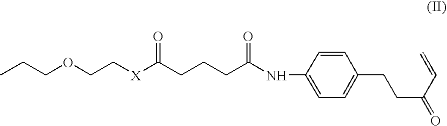

The reactivity of these molecules is comparable to analogous diketones as shown in formula (III). However, the diketones are directly reactive and do not require activation, which raises the possibility of nonspecific reactions with lysine residues other than the lysine residues in the active site, as well as nonspecific reactions with other nucleophiles.

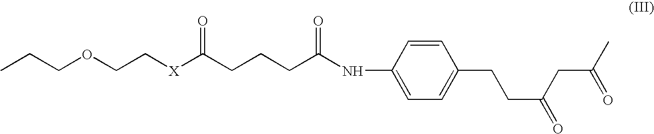

In one alternative, the proadapter comprises a molecule of formula (I) wherein X is NH and n is 2. In another alternative, the proadapter comprises a molecule of formula (I) wherein X is —CH₂NH and n is 3.

In another alternative, the proadapter is a molecule selected from the group consisting of:

(a) a molecule of formula (IV); and

(b) a derivative of a molecule of formula (IV) in which the molecule is substituted with one to five substituents each independently selected from the group consisting of lower alkyl, hydroxyl, alkoxy, and halo and such that the derivative substantially retains the activity with the catalytic moiety of the underivatized molecule.

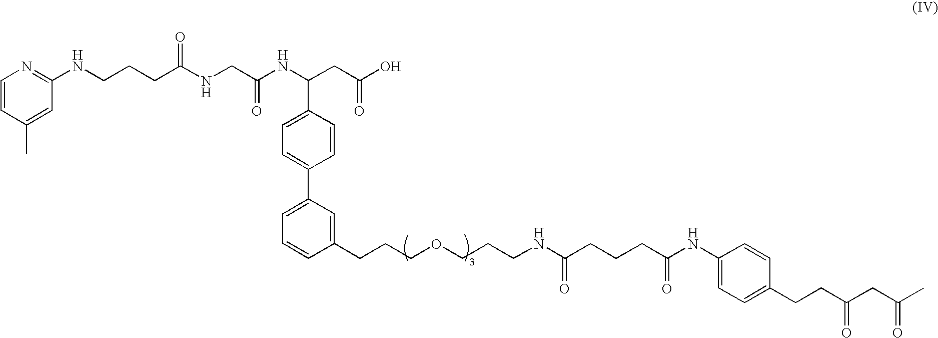

Typically, in this alternative, the proadapter is a molecule of formula (V). This is an analogue to the diketone of formula (VI). The reactivity of the proadapter of formula (V) bears the same relationship to the reactivity of the diketone of formula (VI) as does the proadapter of formula (I) to the diketone of formula (III), described above.

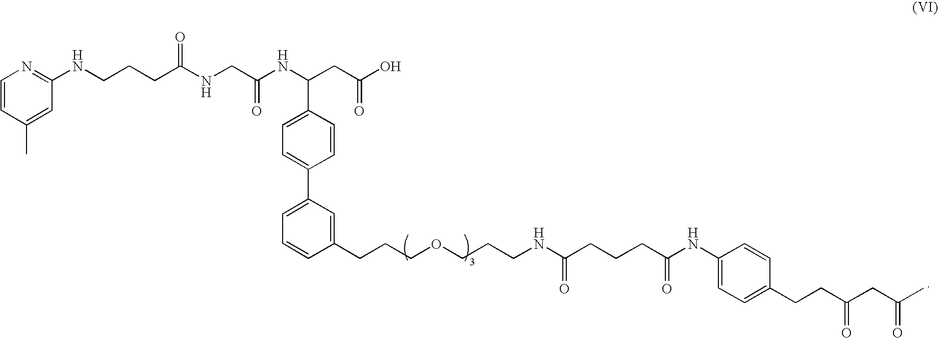

In this alternative, the proadapter is then converted into an adapter of formula (VII) or a corresponding derivative of formula (VII) by the retro-aldol reaction to yield a vinyl ketone reactive with the lysine residue of the active site of the catalytic moiety. This proadapter is described in Example 2 and is particularly suitable for targeting the integrin αᵥβ₆ that is expressed on several cancer cell lines.

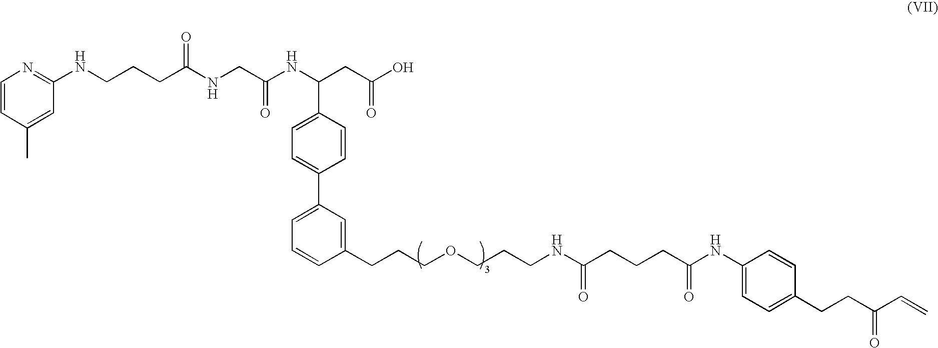

The proadapter is covalently coupled to the signal module. Methods for covalently coupling the proadapter to the signal module are well known in the art. Such methods are described, for example, in U.S. Patent Application Publication No. 2003/0129188 by Barbas et al., in U.S. Patent Application Publication No. 2003/0190676 by Barbas et al., and in U.S. Patent Application Publication No 2003/0175921 by Barbas et al., all incorporated herein by this reference. Such conjugation methods are known in the art. Additional conjugation methods for coupling proadapters to signal modules are described in Example 1, below. Typically, in this approach, signal modules that are protected with the Boc residue are deprotected and then reacted in a condensation reaction with an appropriate N-hydroxysuccinimide ester.

In one alternative, the signal module specifically targets an integrin. Integrins are heterodimeric transmembrane glycoprotein complexes that function in cellular adhesion events and signal transduction processes. Integrin αᵥβ₃ is expressed on numerous cells and has been shown to mediate several biologically relevant processes, including adhesion of osteoclasts to bone matrix, migration of vascular smooth muscle cells, and angiogenesis. Integrin αᵥβ₃ antagonists likely have use in the treatment of several human diseases, including diseases involving neovascularization, such as rheumatoid arthritis, cancer, and ocular diseases.

Suitable signal modules specific for integrins include RGD peptides or peptidomimetics or non-RGD peptides or peptidomimetics. As used herein, reference to “Arg-Gly-Asp peptide” or “RGD peptide” is intended to refer to a peptide having one or more Arg-Gly-Asp containing sequence which may function as a binding site for a receptor of the “Arg-Gly-Asp family of receptors”, e.g., an integrin. Integrins, which comprise an alpha and a beta subunit, include numerous types including, α₁β₁, α₂β₁, α₃β₁, α₄β₁, α₅β₁, α₆β₁, α₇β₁, α₈β₁, α₉β₁, α₆β₄, α₄β₇, α_{D}β₂, α_{L}β₂, α_{M}β₂, αᵥβ₁, αᵥβ₃, αᵥβ₅, αᵥβ₆, αᵥβ₆, αₓβ₂, α_{IIb}β₃, α_{IELb}β₇, and the like. The sequence RGD is present in several matrix proteins and is the target for cell binding to matrix by integrins. Platelets contain a large amount of RGD-cell surface receptors of the protein GP II_{b}/IIIₐ, which is primarily responsible, through interaction with other platelets and with the endothelial surface of injured blood vessels, for the development of coronary artery thrombosis. The term RGD peptide also includes amino acids that are functional equivalents (e.g., RLD or KGD) thereof provided they interact with the same RGD receptor. Peptides containing RGD sequences can be synthesized from amino acids by means well known in the art, using, for example, an automated peptide synthesizer, such as those manufactured by Applied Biosystems, Inc., Foster City, Calif.

As used herein, “non-RGD peptide” refers to a peptide that is an antagonist or agonist of integrin binding to its ligand (e.g. fibronectin, vitronectin, laminin, coliagen etc.) but does not involve an RGD binding site. Non-RGD integrin peptides are known for αᵥβ₃ (see, e.g., U.S. Pat. Nos. 5,767,071 and 5,780,426) as well as for other integrins such as αᵥβ₁ (VLA-4) or a4 (7 (see, e.g., U.S. Pat. No. 6,365,619; Chang et al., Bioorganic & Medicinal Chem Lett, 12:159-163 (2002); Lin et al., Bioorganic & Medicinal Chem Lett, 12:133-136 (2002)), and the like.

An integrin signal module may be a peptidomimetic agonist or antagonist, which preferably is a peptidomimetic agonist or antagonist of an RGD peptide or non-RGD peptide. As used herein, the term “peptidomimetic” is a compound containing non-peptidic structural elements that are capable of mimicking or antagonizing the biological action(s) of a natural parent peptide. A peptidomimetic of an RGD peptide is an organic molecule that retains similar peptide chain pharmacophore groups of the RGD amino acid sequence but lacks amino acids or peptide bonds in the binding site sequence. Likewise, a peptidomimetic of a non-RGD peptide is an organic molecule that retains similar peptide chain pharmacophore groups of the non-RGD binding site sequence but lacks amino acids or peptide bonds in the binding site sequence. A “pharmacophore” is a particular three-dimensional arrangement of functional groups that are required for a compound to produce a particular response or have a desired activity. The term “RGD peptidomimetic” is intended to refer to a compound that comprises a molecule containing the RGD pharmacophores supported by an organic/non-peptide structure. It is understood that an RGD peptidomimetic (or non-RGD peptidomimetic) may be part of a larger molecule that itself includes conventional or modified amino acids linked by peptide bonds.

RGD peptidomimetics are well known in the art, and have been described with respect to integrins such as GPII_{b}/IIIₐ, αᵥβ₃ and αᵥβ₅ (See, e.g., Miller et al., J. Med. Chem. 2000, 43:22-26, and International Pat. Publications WO 0110867, WO 9915178, WO 9915170, WO 9815278, WO 9814192, WO 0035887, WO 9906049, WO 9724119 and WO 9600730; see also Kumar et al., Cancer Res. 61:2232-2238 (2000)), all of which are incorporated herein by this reference. Many such compounds are specific for more than one integrin. RGD peptidomimetics are generally based on a core or template (also referred to as “fibrinogen receptor antagonist template”), to which are linked by way of spacers to an acidic group at one end and a basic group at the other end of the core. The acidic group is generally a carboxylic acid functionality while the basic group is generally a N-containing moiety such as an amidine or guanidine. Typically, the core structure adds a form of rigid spacing between the acidic moiety and the basic nitrogen moiety, and contains one or more ring structures (e.g., pyridine, indazole, etc.) or amide bonds for this purpose. For a fibrinogen receptor antagonist, generally, about twelve to fifteen, more preferably thirteen or fourteen, intervening covalent bonds are present (via the shortest intramolecular path) between the acidic group of the RGD peptidomimetic and a nitrogen of the basic group. The number of intervening covalent bonds between the acidic and basic moiety is generally shorter, two to five, preferably three or four, for a vitronectin receptor antagonist. The particular core may be chosen to obtain the proper spacing between the acidic moiety of the fibrinogen antagonist template and the nitrogen atom of the pyridine. Generally, a fibrinogen antagonist will have an intramolecular distance of about 16 Å (1.6 nm) between the acidic moiety (e.g., the atom which gives up the proton or accepts the electron pair) and the basic moiety (e.g., which accepts a proton or donates an electron pair), while a vitronectin antagonist will have about 14 Å (1.4 nm) between the respective acidic and basic centers. Further description for converting from a fibrinogen receptor mimetic to a vitronectin receptor mimetic can be found in U.S. Pat. No. 6,159,964, incorporated herein by this reference.

The peptidomimetic RGD core can comprise a 5-11 membered aromatic or nonaromatic mono- or polycyclic ring system containing 0 to 6 double bonds, and containing 0 to 6 heteroatoms chosen from N, O and S. The ring system may be unsubstituted or may be substituted on a carbon or nitrogen atom. Preferred core structures with suitable substituents useful for vitronectin binding include monocyclic and bicyclic groups, such as benzazapine described in WO 98/14192, benzdiazapine described in U.S. Pat. No. 6,239,168, and fused tricyclics described in U.S. Pat. No. 6,008,213, all of which are incorporated herein by this reference.

U.S. Pat. No. 6,159,964, incorporated herein by this reference, contains an extensive list of references in Table 1 of that document which disclose RGD peptidomimetic core structures (referred to as fibrinogen templates) which can be used for preparing RGD peptidomimetics. Preferred vitronectin RGD and fibronectin RGD peptidomimetics are disclosed in U.S. Pat. Nos. 6,335,330; 5,977,101; 6,088,213; 6,069,158; 6,191,304; 6,239,138; 6,159,964; 6,117,910; 6,117,866; 6,008,214; 6,127,359; 5,939,412; 5,693,636; 6,403,578; 6,387,895; 6,268,378; 6,218,387; 6,207,663; 6,011,045; 5,990,145; 6,399,620; 6,322,770; 6,017,925; 5,981,546; 5,952,341; 6,413,955; 6,340,679; 6,313,119; 6,268,378; 6,211,184; 6,066,648; 5,843,906; 6,251,944; 5,952,381; 5,852,210; 5,811,441; 6,114,328; 5,849,736; 5,446,056; 5,756,441; 6,028,087; 6,037,343; 5,795,893; 5,726,192; 5,741,804; 5,470,849; 6,319,937; 6,172,256; 5,773,644; 6,028,223; 6,232,308; 6,322,770; and 5,760,028, all of which are incorporated herein by this reference.

Exemplary RGD peptidomimetic integrin targeting agents, such as those shown as compounds 1, 2, and 3 in U.S. Patent Application Publication No. 2003/0129188 by Barbas et al., incorporated herein by this reference, can be used for preparing an intregrin signal module according to the present invention. These compounds are modified or attached to a linker such that they are attached to the proadapter. Other RGD peptidomimetic integrin targeting agents include compound 33 as shown in U.S. Patent Application Publication No. 2003/0129188 by Barbas et al., wherein P and L are carbon or nitrogen. The linker may be R1 or R2 while the R3 group includes a basic group such as an —NH group. In some embodiments, the R3 group is as shown in compounds 1, 2, or 33 of U.S. Patent Application Publication No. 2003/0129188 by Barbas et al. In some embodiments, the R3 group includes a heterocyclic group such a benzimidazole, imidazole, pyridine group, or the like. In some such embodiments, the R3 group is an alkoxy group, such as a propoxy group or the like, that is substituted with a heterocyclic group that is substituted with an alkylamine group, such as a methylamino group or the like, whereas in other embodiments, the R3 group is an alkoxy group, such as a propoxy group or the like, substituted with a heterocyclylamino group, such as with a pyridinylamino group or the like such as a 2-pyridinylamino group. In other embodiments R3 is a group of formula —C(═O)Rb where Rb is selected from —N(alkyl)-alkyl-heterocyclic groups such as —N(Me)-CH₂-benzimidazole groups and the like.

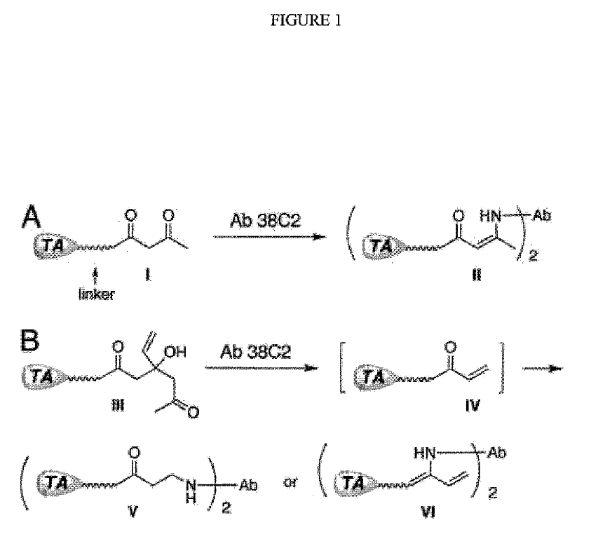

Other exemplary integrin peptidomimetic signal modules and a peptide signal module are shown in FIG. 1 of U.S. Patent Application Publication No 2003/0129188 by Barbas et al. The linker may be any of R₁, R₂, R₃, While R₄ may be a linker or a hydrolyzable group such as alkyl, alkenyl, alkynyl, oxoalkyl, oxoalkenyl, oxoalkynyl, aminoalkyl, aminoalkenyl, aminoalkynyl, sulfoalkyl, sulfoalkenyl, or sulfoalkynyl group, phosphoalkyl group, phosphoalkenyl group, phosphoalkynyl group, and the like, as described in U.S. Patent Application Publication No. 2003/0129188 by Barbas et al. One of skill in the art will readily appreciate that other integrin agonist and antagonist mimetics can also be used in signal modules of the present invention.

In one alternative, the target molecule to which the signal module binds is a non-immunoglobulin molecule. Alternatively, the signal module can bind to an immunoglobulin molecule, In general, the target molecule can be any type of molecule including organic, inorganic, protein, lipid, carbohydrate, nucleic acid and the like.

Still other signal modules are within the scope of the invention. These include the modified T-20 peptide having the amino acid sequence N-Acetyl-YTSLIHSLIEESQNQQEKNEQELLELDKWASLWNWFC (SEQ ID NO: 1). This peptide is a derivative of the peptide T-20, N-Acetyl-YTSLIHSLIEESQNQQEKNEQELLELDKWASLWNWF (SEQ ID NO: 2), with an additional carboxyl-terminal cysteine. T-20 is a synthetic peptide corresponding to a region of the transmembrane subunit of the HIV-1 envelope protein, and that blocks cell fusion and viral entry at concentrations of less than 2 ng/ml in vitro. When administered intravenously, T-20 (monotherapy), the peptide decreases plasma HIV RNA levels demonstrating that viral entry can be successfully blocked in vivo. Administration of T-20 provides potent inhibition of HIV replication comparable to anti-retroviral regimens approved at present (Kilby et al., Nat. Med., 1998, 4(11):1302-7, incorporated herein by this reference). This peptide drug suffers from a short half-life in vivo of approximately 2 hours. The thiol-labeled peptide is suitable for use as a targeting module and can be used to inhibit HIV-1 entry and infection, as described in Example 8 of U.S. Patent Application Publication No. 200310129188 by Barbas et al. In addition to peptides that target the envelope proteins of HIV-1, a number of small-molecules that bind the envelope proteins have been described. For example, the betulinic acid derivative IC9564 is a potent anti-human immunodeficiency virus (anti-H IV) compound that can inhibit both HIV primary isolates and laboratory-adapted strains. Evidence suggests that HIV-1 gp120 plays a key role in the anti-HIV-1 activity of IC₉₅₆₄ (Holz-Smith et al., Antimicrob Agents Chemother., 2001, 45(1):60-6, incorporated herein by this reference). Preparing a chemically programmed antibody in which IC9564 is the signal module is expected to have increased activity over IC9564 itself by increasing valency, half-life, and by directing immune killing of HIV-1 infected cells based on the constant region of the antibody chosen. Similarly, recent X-ray crystallographic determination of the HIV-1 envelope glycoprotein gp41 core structure opened up a new avenue to discover antiviral agents for chemotherapy of HIV-1 infection and AIDS. Compounds with the best fit for docking into the hydrophobic cavity within the gp41 core and with maximum possible interactions with the target site can also be improved by addition of a diketone arm and covalent linkage to an antibody. Several compounds of this class have been identified (Debnath et al., J Med. Chem., 1999, 42(17):3203-9, incorporated herein by this reference). These peptides and their derivatives can be used as signal modules in the same manner as cysteine-labeled T-20.

Another signal module is a nilutamide analog that targets the androgen receptor, as described in P. S. Cogan & T. H. Koch, “Rational Design and Synthesis of Androgen Receptor-Targeted Nonsteroidal Anti-Androgen Ligands for the Tumor-Specific Delivery of a Doxorubicin-Formaldehyde Conjugate,” J. Med. Chem. 46: 5258-5270 (2003), incorporated herein by this reference.

The target molecule bound by the signal module is preferably a biomolecule such as a protein, carbohydrate, lipid or nucleic acid. The target molecule can be associated with a cell (“cell surface expressed”), or other particle (“particle surface expressed”) such as a virus, or may be extracellular. If associated with a cell or particle, the target molecule is preferably expressed on the surface of the cell or particle in a manner that allows the targeting agent of the targeting compound to make contact with the surface receptor from the fluid phase of the body.

In some preferred embodiments, the target molecule bound by the signal module is predominantly or exclusively associated with a pathological condition or diseased cell, tissue or fluid. Thus, a chemically programmed antibody according to the present invention can be used to deliver the signal module to a diseased tissue by targeting the cell, an extracellular matrix biomolecule or a fluid biomolecule. Exemplary target molecules disclosed hereinafter in the Examples of U.S. Patent Application Publication No. 2003/0129188 by Barbas et al. include integrins (Example 1), cytokine receptors (Examples 2, 3 and 7), cytokines (Example 4), vitamin receptors (Example 5), cell surface enzymes (Example 6), and HIV-1 virus and HIV-1 virus infected cells (Examples 8 and 11), and the like.

In other preferred embodiments, the target molecule is associated with an infectious agent and is expressed on the surface of a microbial cell or on the surface of a viral particle. As such, chemically programmed antibodies according to the present invention in which the signal module can bind to the cell surface expressed or particle expressed infectious agent can be used as an anti-microbial, by targeting microbial agents inside the body or on the surface (e.g., skin) of an individual. In the latter case, the invention compound can be applied topically.

Chemically programmed antibodies according to the present invention where the signal module is specific for a microbial target molecule also can be used as an anti-microbial agent in vitro. Accordingly, a method of reducing the infectivity of microbial cells or viral particles present on a surface is provided. Some methods include contacting the surface of a microbial cell or viral particle with an effective amount of the chemically programmed antibody. The chemically programmed antibody in such methods includes a signal module specific for a receptor on the microbial cell or virus particle. Applicable surfaces are any surfaces in vitro such as a counter top, condom, and the like.

Another preferred target molecule for chemically programmed antibodies of the invention is prostate specific antigen (PSA), a serine protease that has been implicated in a variety of disease states including prostate cancer, breast cancer and bone metastasis. Specific inhibitors of PSA which bind to the active site of PSA are known. See Adlington et al., J. Med. Chem., 2001, 44:1491-1508 and WO 98/25895 to Anderson, incorporated herein by this reference. A specific inhibitor of PSA is shown in U.S. Patent Application Publication No. 2003/0129188 by Barbas et al. as compound 34.

A signal module, in addition to its ability to bind a target molecule, may be characterized in having one or more biological activities, each activity characterized as a detectable biological effect on the functioning of a cell organ or organism. Thus, in addition to being a signal module, such compounds can be considered biological agents. For example, the integrin targeting modules shown as compounds 1, 2, 3 and 33 in U.S. Patent Application Publication No. 2003/0129188 by Barbas et al., or derivatives of these molecules possessing a hydroxylamine group or other group capable of reacting with an aldehyde-containing amino acid as described above, above not only target an integrin, but have integrin antagonist biological activity. In some embodiments, however, a signal module may be a pure binding agent without biological activity.

The signal module can be attached to the proadapter either directly or through a linker. Depending on the chemical structure and reactivity of the signal module, the linker can be a peptide or nonpeptide linker. If a peptide linker, it is typically from about 3 to about 50 amino acid residues in length, more typically from about 5 to 25 amino acid residues in length. Appropriate linkers and reactions that can be used to attach the signal module to the linker are described, for example, in U.S. Patent Application Publication No. 2003/0129188 by Barbas et al., in U.S. Patent Application Publication No. 2003/0190676 by Barbas et al., and in U.S. Patent Application Publication No. 2003/0175921 by Barbas et al., all incorporated herein by this reference. Such reactions are also described in G. T. Hermanson, “Bioconjugate Techniques” (Academic Press, San Diego, 1996), and in S. S. Wong, “Chemistry of Protein Conjugation and Cross-Linking” (CRC Press, Boca Raton, Fla., 1993), both also incorporated herein by this reference. These linkers can make use of the reaction between biotin or biotin analogues and avidin or streptavidin. These linkers and reactions are generally known in the art.

Particular signal modules may or may not possess biological activity depending on the context of their use. For example, the therapeutic drug doxorubicin, which is a DNA intercalator, can be a targeting module for double stranded DNA when the drug is covalently linked to an antibody and applied to DNA in a cell-free form. Doxorubicin, however, may not be considered a signal module with respect to a cell while the drug is covalently linked to an antibody unless the compound can be taken up by the cell. In the latter case, doxorubicin may have biological activity following uptake if the drug can access DNA in the cell nucleus.

Biological agent functional components include, but are not limited to, small molecule drugs (a pharmaceutical organic compound of about 5,000 daltons or less), organic molecules, proteins, peptides, peptidomimetics, glycoproteins, proteoglycans, lipids, glycolipids, phospholipids, lipopolysaccharides, nucleic acids, proteoglycans, carbohydrates, and the like. Biological agents may be anti-neoplastic, anti-microbial, a hormone, an effector, and the like. Such compounds include well known therapeutic compounds such as the anti-neoplastic agents paclitaxel, daunorubicin, doxorubicin, caminomycin, 4′-epiadriamycin, 4-demethoxy-daunomycin, 11-deoxydaunorubicin, 13-deoxydaunorubicin, adriamycin-14-benzoate, adriamycin-14-octanoate, adriamycin-14-naphthalene acetate, vinbiastine, vincristine, mitomycin C, N-methyl mitomycin C, bleomycin A₂, dideazatetrahydrofolic acid, aminopterin, methotrexate, colchicine and cisplatin, and the like. Anti-microbial agents include aminoglycosides including gentamicin, antiviral compounds such as rifampicin, 3′-azido-3′-deoxythymidine (AZT) and acylovir, antifungal agents such as azoles including fluconazole, macrolides such as amphotericin B, and candicidin, anti-parasitic compounds such as antimonials, and the like. Hormones may include toxins such as diphtheria toxin, cytokines such as CSF, GSF, GMCSF, TNF, erythropoietin, immunomodulators or cytokines such as the interferons or interleukins, a neuropeptide, reproductive hormone such as HGH, FSH, or LH, thyroid hormone, neurotransmitters such as acetylcholine, hormone receptors such as the estrogen receptor. Also included are non-steroidal anti-inflammatories such as indomethacin, salicylic acid acetate, ibuprofen, sulindac, piroxicam, and naproxen, and anesthetics or analgesics. Also included are radioisotopes such as those useful for imaging as well as for therapy.

Biological agent functional components for use as signal modules in chemically programmed antibodies according to the invention can be naturally occurring or synthetic. Biological agents can be biologically active in their native state, or be biologically inactive or in a latent precursor state, i.e., a prodrug, and acquire biological or therapeutic activity when a portion of the biological agent is hydrolyzed, cleaved or is otherwise modified. The prodrug can be delivered at the surface of a cell or intracellularly using antibody targeting compounds of the invention where it can then be activated. In this regard, the biological agent can be a “prodrug,” meaning that prodrug molecules capable of being converted to drugs (active therapeutic compounds) by certain chemical or enzymatic modifications of their structure. In the prodrug approach, site-specific drug delivery can be obtained from tissue-specific activation of a prodrug, which is the result of metabolism by an enzyme that is either unique for the tissue or present at a higher concentration (compared with other tissues); thus, it activates the prodrug more efficiently.

In another alternative, the signal module can primarily function as a label for the target; for example, the signal module can be a fluorescent, chemiluminescent, or bioluminescent molecule. The signal module can also incorporate a direct label, such as a colloidal gold label. The signal module can also be any molecule incorporating a detectable radioisotope. As another alternative, the signal module can be a protein, such as an enzyme that catalyzes a reaction that produces a detectable product. In another alternative, the signal module can be a protein that is detected by the use of a secondary labeled antibody that specifically binds the targeting module. The product can be detectable calorimetrically, by fluorescence, by chemiluminescence, by bioluminescence, or by its reaction with another molecule. An example is the hydrolytic enzyme β-galactosidase. The signal module can also be detectable by a biological property, such as drug resistance. Accordingly, the signal module can be or include a protein such as an enzyme, another antibody or portion thereof, or a receptor. Receptors can include thrombospondin receptors, such as CD36. In still another alternative, the signal module can be a nucleic acid sequence as the term “nucleic acid sequence” is defined above. The nucleic acid sequence can hybridize specifically to a cellular or viral nucleic acid sequence such as DNA or mRNA. The nucleic acid sequence can be an antisense sequence as is known in the art. Therefore, as used herein, the term “signal module” used as described above to include molecules that have targeting or labeling activity as described above, unless otherwise further specified.

The chemically programmed antibody can have other proteins, peptides, or protein domains to fused to it to generate a larger fusion protein. For example, other proteins, peptides, or domains from other proteins, can be fused to the carboxyl terminus of the Fc where the catalytic moiety includes therein an intact antibody molecule. These proteins can include, but are not limited to, a cytokine like IL-2, or even another antibody fragment like a scFv. These proteins can also include enzymes or receptors, as well as peptides such as a polyhistidine or a FLAG purification tag.

The method described above can be performed in vitro. When the method is performed in vitro, the resulting chemically programmed antibody is typically administered to a subject in need of treatment with the chemically programmed antibody in a pharmaceutical composition as described further below.

In another alternative, the method described above can be performed in vivo, as described further in Example 1. When the method is performed in vivo, the catalytic moiety can be administered to the subject in a pharmaceutical composition. Alternatively, a nucleic acid sequence, typically a DNA sequence, encoding the catalytic moiety can be administered to the subject in a pharmaceutical composition so that transcription and/or translation of the nucleic acid sequence generates the catalytic moiety.

Typically, the catalytic moiety is an intact antibody molecule. However, it is also possible to produce a chemically programmed antibody using the Fab fragment of an antibody molecule. However, particularly when it is desired to make use of the effector functions mediated by the Fc portion of an intact antibody molecule, it is generally preferred that the catalytic moiety is an intact antibody molecule. However, another alternative includes some but not all of the Fc portion of the antibody molecule.

Another aspect of the present invention is a chemically programmed antibody formed by the method described above. In one preferred alternative, the antibody incorporates a 38C2 catalytic antibody, as described further in Example 1. In another alternative, the antibody incorporates a Fab fragment of 38C2 catalytic antibody. In this chemically programmed antibody, the proadapter and the signal module are as described above and include the possible alternatives described above with respect to the method.

Methods for the isolation of chemically programmed antibodies and catalytic moieties are well known in the art and need not be described further in detail herein. For example, methods such as precipitation with salts such as ammonium sulfate, ion exchange chromatography, gel filtration, affinity chromatography, electrophoresis, isoelectric focusing, isotachophoresis, chromatofocusing, and other techniques are well known in the art and are described in R. K. Scopes, “Protein Purification: Principles and Practice” (3^{rd} ed., Springer-Veriag, New York, 1994).

In one preferred alternative, a chemically programmed antibody according to the invention whose signal module targets an appropriate integrin, such as αᵥβ₃, αᵥβ₅, or αᵥβ₆, is internalized in cells via an integrin-dependent endocytosis pathway. This allows the use of such chemically programmed antibodies for drug delivery in circumstances where it is required that the drug be internalized.

Accordingly, another aspect of the present invention is a proadapter useful for the production of a chemically programmed antibody according to the present invention as described above. Particularly useful proadaptors are described in Example 1 and are of formula (I), especially compounds of formula (I) in which X is NH and n is 2, or in which X is —CH₂NH and n is 3, as well as derivatives thereof. Additional proadapters are described in Example 2 and are of formula (V), as well as derivatives thereof.

Still another aspect of the present invention is a conjugate comprising:

(1) a proadapter comprising a molecule selected from the group consisting of:
(a) a molecule of formula (I) wherein X is selected from the group consisting of NH and —CH₂NH and n is 2 or 3; and
(b) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule; and
(c) a signal module covalently linked to the proadapter.

Suitable signal modules are as described above. Typically, the proadapter is a molecule of formula (I).

Particular conjugates according to the present invention are those of formula (IV) in which X is NH and n is 2, or of formula (IV) in which X is —CH₂NH and n is 3. These are described in more detail in Example 1.

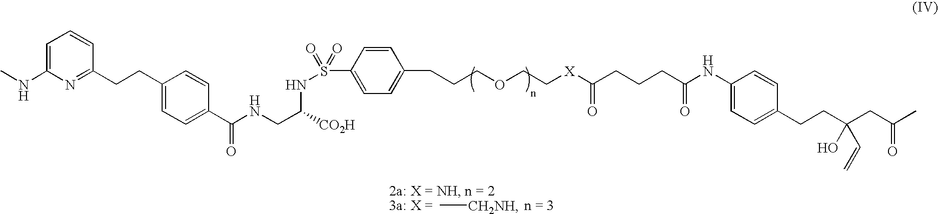

Alternatively, conjugates according to the present invention can incorporate a proadapter of formula (V) or a derivative thereof. These conjugates comprise:

(a) a proadapter comprising a molecule selected from the group consisting of:

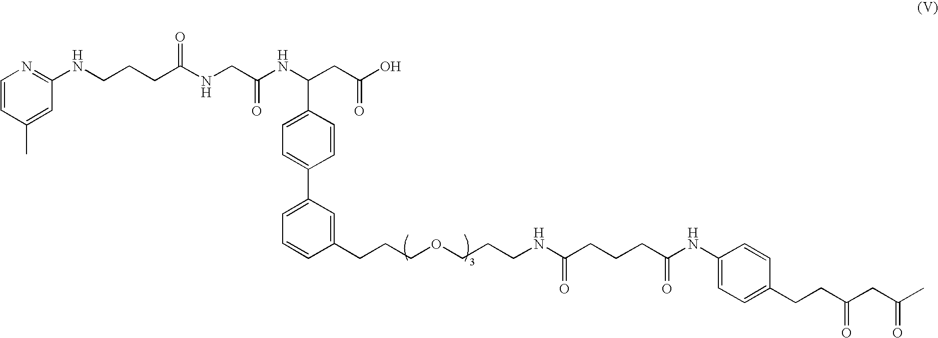

(i) a molecule of formula (V); and
(ii) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule; and

(b) a signal module covalently linked to the proadapter.

Suitable signal modules are as described above. In this alternative, typically the proadapter is a molecule of formula (V).

Another aspect of the present invention is a pharmaceutical composition including therein a chemically programmed antibody according to the present invention as described above. The pharmaceutical composition comprises:

(1) a chemically programmed antibody according to the present invention in a therapeutically effective quantity; and

(2) a pharmaceutically acceptable carrier.

Accordingly, the chemically programmed antibodies can be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, sodium citrate, and the like.

Alternatively, chemically programmed antibodies can be encapsulated, tableted or prepared in an emulsion or syrup for oral administration. Pharmaceutically acceptable solid or liquid carriers can be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation may be in the form of a syrup, elixir, emulsion, or an aqueous or non-aqueous suspension. For rectal administration, the invention compounds may be combined with excipients such as cocoa butter, glycerin, gelatin or polyethylene glycols and molded into a suppository.

Chemically programmed antibodies according to the present invention can be formulated to include other medically useful drugs or biological agents. The chemically programmed antibodies also can be administered in conjunction with the administration of other drugs or biological agents useful for treatment of the disease or condition that chemically programmed antibodies according to the present invention are administered to treat. Such diseases and conditions include, but are not limited to, cancer, including, but not limited to, metastatic breast cancer and metastatic melanoma.

As employed herein, the phrase “a therapeutically effective quantity,” refers to a dose sufficient to provide concentrations high enough to impart a beneficial effect on the recipient thereof. The use of terms such as “therapeutically effective quantity” or similar terminology does not imply the existence of a cure for a disorder being treated by the administration of chemically programmed antibodies according to the present invention or compositions including chemically programmed antibodies according to the present invention. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective quantity” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. Dosage levels typically fall in the range of about 0.001 up to 100 mg/kg/day; with levels in the range of about 0.05 up to 10 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The composition may be administered as a bolus, or slowly infused.

The administration of a chemically programmed antibody to an immunocompetent individual may result in the production of antibodies against the labeled protein, depending on the origin of the components of the chemically programmed antibody. Such antibodies may be directed to the Fc portion of the antibody itself or to other regions of the chemically programmed antibody, such as the signal module or any linker used in the production of the chemically programmed antibody. Reducing the immunogenicity of the chemically programmed antibody can be addressed by methods well known in the art such as by attaching long chain polyethylene glycol (PEG)-based spacers, and the like, to the antibody-targeting agent. Long chain PEG and other polymers are known for their ability to mask foreign epitopes, resulting in the reduced immunogenicity of therapeutic proteins that display foreign epitopes (Katre et al., 1990, J, Immunol. 144, 209-213; Francis et al., 1998, Int. J. Hematol. 68, 1-18, incorporated herein by this reference). Alternatively, or in addition, the individual administered the labeled protein may be administered an immunosuppressant such as cyclosporin A, anti-CD3 antibody, and the like, as appropriate to the medical status of the patient and the condition being treated.

Another aspect of pharmaceutical compositions according to the present invention is a pharmaceutical composition for administration of the catalytic moiety itself, such as a catalytic antibody. This is particularly suitable when it is intended to form the chemically programmed antibody in vivo, as discussed above. Therefore, such a pharmaceutical composition comprises:

(1) a catalytic moiety that can form a chemically programmed antibody according to the present invention in a therapeutically effective quantity; and

(2) a pharmaceutically acceptable carrier.

As an alternative to the administration of the catalytic moiety for in vivo formation of a chemically programmed antibody according to the present invention, a nucleic acid that encodes the catalytic moiety can alternatively be administered in the pharmaceutical composition. Therefore, such a pharmaceutical composition comprises:

(1) a therapeutically effective quantity of a nucleic acid encoding a catalytic moiety that can form a chemically programmed antibody according to the present invention; and

(2) a pharmaceutically acceptable carrier.

Typically, the nucleic acid is DNA.

Another aspect of the invention is a method of treatment of a disease or condition treatable by the administration of a chemically programmed antibody according to the present invention.

In one alternative of this aspect, the method comprises administration of a therapeutically effective quantity of a chemically programmed antibody according to the present invention to treat a disease or condition treatable by the administration of the chemically programmed antibody. The disease or condition can be, but is not limited to, cancer. The cancer can be, but is not limited to, metastatic melanoma or metastatic breast cancer, in which case the treatment results in the prevention of metastasis. The therapeutically effective quantity of the chemically programmed antibody can be administered alone, or in a pharmaceutical composition as described above. In this alternative, the chemically programmed antibody is prepared in vitro and then administered.

In another alternative, the chemically programmed antibody is formed in vivo and the catalytic moiety and the proadaptor are administered as two components. In general this method comprises the steps of:

(1) administering a therapeutically effective quantity of a catalytic moiety that can form a chemically programmed antibody according to the present invention when reacted with a proadaptor,

(2) administering a therapeutically effective quantity of a proadaptor that can form a chemically programmed antibody according to the present invention when reacted with the catalytic moiety; and

(3) reacting the catalytic moiety and proadaptor in vivo to form the chemically programmed antibody in a therapeutically effective quantity to treat the disease or condition.

Again, either or both of the catalytic moiety or proadaptor can be administered alone or in a pharmaceutical composition. Both the catalytic moiety and the proadaptor can be administered in the same pharmaceutical composition for in vivo formation of the chemically programmed antibody.

In another alternative, the catalytic moiety is produced by transcription and/or translation of an administered nucleic acid, typically DNA, that encodes the catalytic moiety. Accordingly, this method comprises:

(1) administering a therapeutically effective quantity of a nucleic acid encoding a catalytic moiety that can form a chemically programmed antibody according to the present invention when reacted with a proadaptor;

(2) administering a therapeutically effective quantity of a proadaptor that can form a chemically programmed antibody according to the present invention when reacted with the catalytic moiety; and

(3) reacting the catalytic moiety and proadaptor in vivo to form the chemically programmed antibody in a therapeutically effective quantity to treat the disease or condition.

Typically, the nucleic acid administered is DNA.

DNA sequences encoding the catalytic moiety of the invention, including muteins as described above, can be obtained by several methods. For example, the DNA can be isolated using hybridization procedures that are well known in the art. These include, but are not limited to: (1) hybridization of probes to genomic or cDNA libraries to detect shared nucleotide sequences; (2) antibody screening of expression libraries to detect shared structural features; and (3) synthesis by the polymerase chain reaction (PCR), RNA sequences of the invention can be obtained by methods known in the art (See, for example, Current Protocols in Molecular Biology, Ausubel, et al., Eds., 1989).

The development of specific DNA sequences encoding catalytic moieties of the invention can be obtained by: (1) isolation of a double-stranded DNA sequence from the genomic DNA; (2) chemical manufacture of a DNA sequence to provide the necessary codons for the polypeptide of interest; and (3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA. Of these three methods for developing specific DNA sequences for use in recombinant procedures, the isolation of genomic DNA is the least common. This is especially true when it is desirable to obtain the microbial expression of mammalian polypeptides due to the presence of introns. The synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the formation of cDNA sequences. Among the standard procedures for isolating cDNA sequences of interest is the formation of plasmid-carrying cDNA libraries which are derived from reverse transcription of mRNA which is abundant in donor cells that have a high level of genetic expression. When used in combination with polymerase chain reaction technology, even rare expression products can be clones. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucleic Acid Research 11:2325, 1983, incorporated herein by this reference).

With respect to nucleotide sequences that are within the scope of the invention, all nucleotide sequences encoding the catalytic moieties that are embodiments of the invention as described are included in nucleotide sequences that are within the scope of the invention. This further includes all nucleotide sequences that encode polypeptides according to the invention that incorporate conservative amino acid substitutions as defined above.

Nucleic acid sequences of the present invention further include nucleic acid sequences that are at least 95% identical to the sequences above, with the proviso that the nucleic acid sequences retain the activity of the sequences before substitutions of bases are made, including any activity of proteins that are encoded by the nucleotide sequences and any activity of the nucleotide sequences that is expressed at the nucleic acid level, such as the binding sites for proteins affecting transcription. Preferably, the nucleic acid sequences are at least 97.5% identical. More preferably, they are at least 99% identical. For these purposes, “identity” is defined according to the Needleman-Wunsch algorithm (S. B. Needleman & C. D. Wunsch, “A General Method Applicable to the Search for Similarities in the Amino Acid Sequence of Two Proteins,” J. Mol. Biol. 48: 443-453 (1970)), incorporated herein by this reference.

Nucleotide sequences encompassed by the present invention can also be incorporated into a vector, including, but not limited to, an expression vector, and used to transfect or transform suitable host cells, as is well known in the art. The vectors incorporating the nucleotide sequences that are encompassed by the present invention are also within the scope of the invention. Host cells that are transformed or transfected with the vector or with polynucleotides or nucleotide sequences of the present invention are also within the scope of the invention. Typically, for treatment purposes, the host cells will be eukaryotic cells, more typically mammalian cells, preferably human cells. However, prokaryotic cells can also serve as host cells.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic systems, and preferably mammalian expression systems, allow for proper post-translational modifications of expressed mammalian proteins to occur.

A variety of host-expression vector systems may be utilized to express the catalytic moiety. These systems are well known in the art and need not be described further here.

The invention is illustrated by the following Examples. These Examples are for illustrative purposes only and are not intended to limit the invention.

### EXAMPLE 1

Monoclonal antibodies are a rapidly growing class of therapeutics for a wide variety of diseases. Some of the advantages of antibodies include their relative lack of nonspecific toxicity, long half-life, and ease of access from patient-derived or synthetic combinatorial antibody libraries. For certain diseases, such as cancer, that antibodies can carry their own effector functions is of prime importance because the antibody specificity directs the killing function endemic to the effector domain, the Fc. It has always been axiomatic in immunochemistry that even though one may desire one or more of the advantageous properties common to all antibodies, due to their clonal nature, each task requires a different antibody. A solution to this problem, namely chemically programmed antibodies (cpAbs), has emerged at the interface of chemistry and biology: One can use different low-molecular-weight targeting agents (programming agents or adapters) to selectively target the same antibody to different sites for different uses (3). This strategy has the advantage that only a single antibody is required for a multiplicity of tasks, and it taps into the unlimited chemical diversity and the specificity that can be engendered by organic synthesis (4). The antibody provides the organic compound a half-life, biodistribution, valency, and effector function that it may not otherwise have.

The cpAb approach that we have reported is unique in that small synthetic molecules or peptides and catalytic mAbs react in a self-assembly process and become linked through a covalent bond. This covalent modification results in the reprogramming of the specificity of the antibody with the binding specificity of the small molecule. The resulting conjugate of small molecule and antibody is a cpAb. Significantly, we have demonstrated that chemical programming of a catalytic antibody can occur both in vitro and in vivo to have a therapeutic effect in disease models (3, 5). Key to this approach is the development of catalytic antibodies that operate using covalent reaction mechanisms (6, 7). mAb 38C2 is an antibody of this type, an aldolase antibody generated by reactive immunization that contains a highly reactive lysine residue that is key to its activity Small molecules or targeting agents are adapted to work in this approach by addition of a reactive tag that the antibody, through its catalytic function, selectively processes to form a covalent link between itself and the programming agent.

Thus, to selectively target the antibody to particular cells, an antibody-reactive tag is linked to a targeting agent that is a ligand for the desired cellular receptor(s). In this Example, we direct catalytic aldolase antibody to the integrin αᵥβ₃. The integrins αᵥβ₃ and αᵥβ₅ are intriguing targets for cancer therapeutics because these receptors are expressed both on a variety of cancers and on the activated endothelial cells of the angiogenic vasculature they induce (8, 9, 10). The results presented here differ from previous studies (3, 4, 5, 11), in that the reactive tags studied here can be considered proadapters as the antibody uses two catalytic steps to generate a stable covalent complex. Our earlier studies in this area focused on the use of reactive tags that provided for reversible enaminone-attachment chemistry. In this new approach, the reactive tag is first catalytically activated by a retro-aldol reaction that unveils a reactive vinyl ketone that is subsequently covalently attached to the antibody through a Michael addition reaction. In this Example, we explore the chemistry, biology, and therapeutic potential of this proadapter strategy and a peptidomimetic targeting agent in cancer.

### Results and Discussion

In our previous reports, we reacted the small-molecule antagonists of αᵥβ₃ and αᵥβ₅ integrins equipped with a diketone linker, such as 1, with the reactive lysine residues in the aldolase antibody 38C2-binding sites to form the corresponding enaminone derivative, II (FIG. 1A) (3, 4, 5, 11).

FIG. 1 is a general schematic diagram showing the formation of cell-targeting antibody constructs based on adapter (A) and proadapter (B) approaches by using a β-diketone-equipped low-molecular weight targeting agent (“signal module”) and acetone adduct of the vinyl ketone-equipped targeting agent respectively (TA=targeting agent).

In the proadapter approach, we anticipated that a targeting agent equipped with a tertiary aldol linker, such as III, would undergo a 38C2-catalyzed retro-aldol reaction (12) to produce an adapter possessing a reactive linker, such as the vinyl ketone IV. The ketone IV would then react as a Michael acceptor with the key nucleophilic amine in the antibody active site to produce conjugate V, a cpAb. Arguably, the intermediate IV could also react with 38C2 forming the corresponding dibenamine complex VI, but in the end that would also be converted to the thermodynamically stable Michael adduct V. In preliminary studies, we found that methylvinyl ketone rapidly inactivated the antibody, indicating that electrophiles of this type would be suitable as reactive tags if their inherent reactivity could be controlled (S. C. S, and S. Abraham, unpublished results). It should be noted that in the structurally and functionally related constructs II and V, the primary differences are the formation and breakdown of the conjugates. Thus, II is reversible, whereas conjugate V is substantially more stable (FIG. 1B).

We observed that both I and III react specifically and quantitatively with the antibody and two equivalents of either compound is sufficient to completely inhibit the catalytic activity of 38C2, indicating that the key lysine residue in each of the two active sites of the antibody are labeled (FIG. 1B). The role of the aldol functionality of III is to mask the reactive vinyl ketone linker that would be expected to react readily with a variety of protein nucleophiles. Because this reactive functionality is only revealed in the active site of the antibody after the retro-aldol reaction, it was anticipated that the vinyl ketone functionality would react with the catalytic lysine as soon as it was unveiled and before dissociating from the reactive site. Loss of catalytic reactivity of 38C2 after incubation of III with antibody supports this hypothesis. Prolinker III, given its inherent inertness before activation, should have potential synthetic advantages because it should be inert to reaction with nucleophilic groups that might be present on targeting agents.

αᵥβ₃ Integrin-Targeting Agents for the Antibody Construct Formation

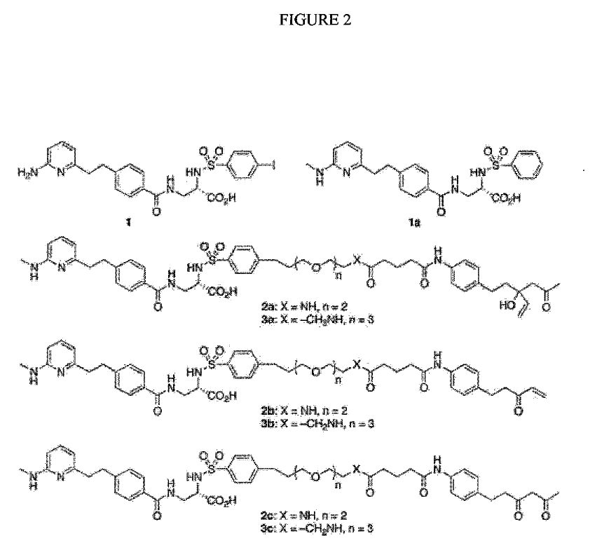

To target 38C2 to αᵥβ₃ integrin using the proadapter approach, two homologous programming agents (2a and 3a) were prepared (see below). Both agents possessed an analog of 1, compound 1a, which binds the αᵥβ₃ integrin with high affinity (FIG. 2), as the targeting agent (13).

FIG. 2 shows structures of the αᵥβ₃ integrin-targeting antagonists equipped with an acetone adduct of a vinyl ketone, a vinyl ketone, or a diketone for chemical programming of the aldolase antibody.

Compound 1a was functionalized with an acetone adduct of the vinyl ketone linkers. This adduct was likely to be a substrate for the retro-aldol reaction catalyzed by the antibody 38C2 to afford 2b or 3b, which should undergo Michael addition with 38C2 to give V (FIG. 1B). As controls for the evaluation of V, the analogous diketone-containing programming agents 2c and 3c were also prepared. The latter compounds should react with 38C2 to give II as shown in FIG. 1A.

### Antibody Construct Formation

To assess the potential of 2a, 3a, 2c, and 3c as programming agents, these compounds were separately mixed with antibody 38C2 at a ratio of 2:1, and the mixtures were incubated at 37° C. for 2 h. A fluorescence assay, based on using methadol as the substrate, was used to assess time-dependent inactivation of the catalytic activity of the antibody (14). Inactivation of aldolase activity should be indicative of modification of the catalytic lysine residue and, thus, chemical programming of the antibody. In the absence of the programming agents, 38C2 rapidly catalyzed the retro-aldol reaction of methadol to produce the fluorescent product 6-methoxy-2-naphthaldehyde. In contrast, the antibody-programming agent constructs were completely inactive, indicating that after conjugation the active site of the catalytic antibody was occupied. (The cpAb 38C2 using targeting agents 2a or 3a were named as 38C2-2b and 38C2-3b, respectively, based on the fact that compounds 2b and 3b were the expected ligands that conjugated with 38C2. Similarly, the analogous chemically programmed 38C2 Fab (or cp38C2Fab) construct obtained from 3a was named as 38C2Fab-3b.) These observations clearly supported the assumption that vinyl ketones, 2b and 3b, produced in situ from their acetone adducts, reacted with the active site of the antibody and also reinforced the previously described construct formation from the analogous diketone compounds 2c and 3c.

The chemical programming of antibody 38C2 using 2b or 3b was also analyzed by MALDI-TOF mass spectrometry for which we used both antibody 38C2 and its Fab fragment. The chemically programmed 38C2 Fab (or cp38C2Fab) was prepared by using a 1:1 mixture of the Fab and compounds 3a or 3c, and their formation was initially analyzed by using the fluorescence assay, as described above. In the mass spectra, chemically programmed 38C2 (i.e., 38C2-3b and 38C2-3c) showed addition of 2 molecules of the programming agents to the average mass of 38C2. Similarly, the analogous cp38C2Fab constructs prepared from 3b or 3c (i.e., 38C2Fab-3b or 38C2F-3c) showed the addition of approximately one molecule of the programming agent to the average mass of the Fab. The average mass peaks 38C2 Fab, 38C2Fab-3b, and 38C2Fab-3c were recorded at 48,410, 49,354, and 49,378, respectively. These observations indicated that the reactive site lysine residues in 38C2 and cp38C2Fabs were labeled specifically compared with any of the many other lysine residues found in the covalent structure of the antibody or Fab.

Binding of Antibody Constructs to αᵥβ₃ Integrin-Expressing Cells

Next, we evaluated binding of the cp38C2 derivatives to αᵥβ₃ integrin-expressing cells by cell flow cytometry. The two cell lines used, MDA-MB435 and MDA-MB-231, are immortalized human breast cancer cell lines, and both cell lines express high levels of αᵥβ₃ integrin (15, 16).

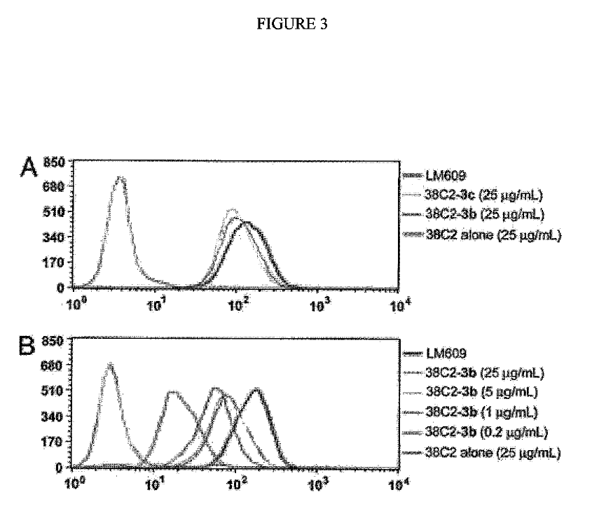

FIG. 3 shows flow cytometry histograms showing the binding of 38C2-3b, 38C2-3c, and 38C2 alone (A) and binding of serial dilutions of 38C2-3b to MDA-MB-231 cells. In A, 38C2-3b and 38C2-3c prepared from 38C2 (1 eq) and 3a or 3c (2 eq) were diluted to 25 μg/ml. In B, the 38C2-3b (25 μg/ml) construct used in A was further diluted 5×, 25×, and 125×. In all experiments, 38C2 alone was used at 25 μg/ml, LM609 was used at 1:100 dilution, and FITC-conjugated goat anti-mouse secondary antibodies were used for detection. The y axis gives the number of events in linear scale, the x axis gives the fluorescence intensity in logarithmic scale.

All cpAb constructs and control anti-integrin antibody LM609 bound efficiently to these cells (FIG. 3A). As expected, antibody 38C2 alone did not bind to these cells. Flow cytometric staining with the cp38C2s, i.e., 38C2-3b and 38C2-3c at 25 and 5 μg/ml, produced profiles with nearly identical fluorescence intensities, but fluorescence decreased considerably at 1 μg/ml and lower concentrations (FIG. 3B). This data implied that 38C2-3b and 38C2-3c provides maximal staining after incubation at a concentration of ≈5 μg/ml (33 nM).

In Vivo Assembly of cpAbs

In previous studies (3, 5), we noted that a diketone linker-equipped integrin antagonist SCS-873 was able to conjugate in vivo with antibody 38C2 and that the resulting cp38C2 had a serum half-life 200 times longer than that of the antagonist itself. The half-life of the SCS-873-based cp38C2 was ≈3 days (3), whereas the half-life determined for 38C2 itself was 4 days (17). Effective in vivo assembly allowed both the antagonist and 38C2 to be administered separately to inhibit the tumor growth in animal models. Here we studied in vivo assembly using the proadapter approach. Key to this assembly is antibody-catalyzed retro-aldolization to provide the vinyl ketone product that could then self-attach to 38C2. Our previous studies concerning 38C2-catalyzed prodrug activation in vivo provides precedence for this approach (18). To evaluate this, we carried out experiments using compound 2a and the conventional diketone 2c as a control. Antibody 38C2 (1 mg in 100 l of PBS buffer) was administered i.v. to three mice followed by i.p. administration of compounds 2a (1 mg in 100 μl of buffer), 2c (1 mg in 100 μl of buffer), or PBS (100 μl). Sera obtained at regular intervals (24, 48, 72, 96, and 168 h) were examined for the presence of cp38C2 by using flow cytometry and MDA-MB231 cells. This study confirmed that the complex, cp38C2, was formed in vivo with both 2a and 2c. Sera from the mouse lacking the targeting agent did not show any binding to the cells. Thus, compound 2a was effectively processed by 38C2 in vivo to form vinyl ketone 2b, which then self-assembled to form cp38C2. The resulting cp38C2-V species (i.e., 38C2-2b) was stable in serum, with a half-life of ≈60 h as analyzed by comparing the mean fluorescence intensity; similar results were observed for the cp38C2-II species (≈60 h for 38C2-2c, as well).

### Cellular Uptake of the Antibody Adapter Constructs

It is well established that αᵥβ₃/αᵥβ₅ integrin-ligand complexes are rapidly internalized via an integrin-dependent endocytosis pathway. Examples of ligands that are effectively internalized include viruses (19) and αᵥ-integrin-blocking mAb. In contrast, internalization of the cyclic synthetic arginine-glycine-aspartate (RGD) motif (cRGD) peptides targeted for αᵥβ₃ takes place by an integrin-independent fluid-phase endocytosis pathway (20). To assess the feasibility of using the integrin-targeting cp38C2 constructs for drug delivery, we evaluated the internalization of the cp38C2 variants. Internalization of 38C2-3b and 38C2-3c into MDA-MB-231 cells was studied. Briefly, 150,000 cells were incubated with 38C2-3b or 38C2-3c (5 μg/ml in cell culture buffer; 100 μl) for 15 min on a coverslip. The cell-38C2-3b and cell-38C2-3c ternary complexes were then fixed by using 2% paraformaldehyde in 0.01 M PBS and incubated with FITC-conjugated goat anti-mouse secondary antibody for 60 min. Similar experiments were carried out by using 38C2 alone as the negative control. The cells were analyzed by using confocal laser scanning microscopy (21). Cells treated with 38C2-3b and 38C2-3c were rapidly internalized probably through an integrin-mediated endocytosis mechanism. If a nonintegrin-mediated or Fc-based internationalization mechanism was operative, fluorescence should have been observed with antibody 38C2 alone.

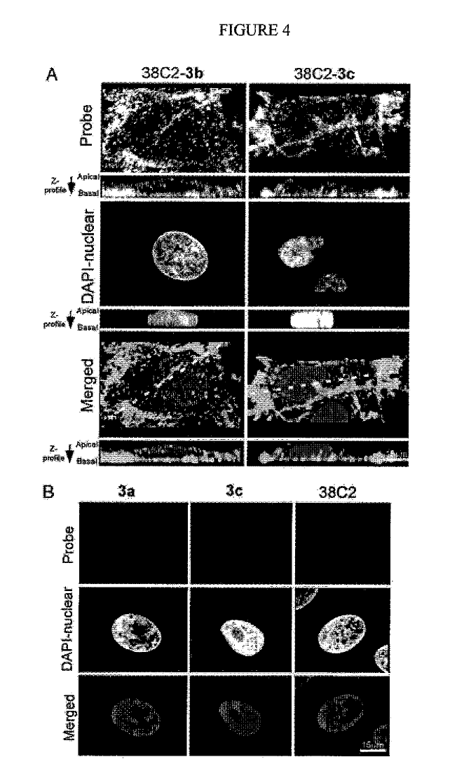

FIG. 4 shows the results of a cell uptake assay using integrin αᵥβ₃-targeting 38C2 constructs (38C2-3b and 38C2-3c) (A) and compounds 3a and 3c and mAb 38C2 in MDA-MB-231 cells (B). Antibody constructs 3802-3b or 38C2-3c and antibody 38C2 alone were used at 5 μg/ml in PBS buffer. Compounds 3a and 3c were used at a 66.7 μM concentration (twice the concentration of 38C2-3b or 38C2-3c). FITC-conjugated goat anti-mouse secondary antibodies were used for detection.

### Prevention of Breast Cancer Metastasis

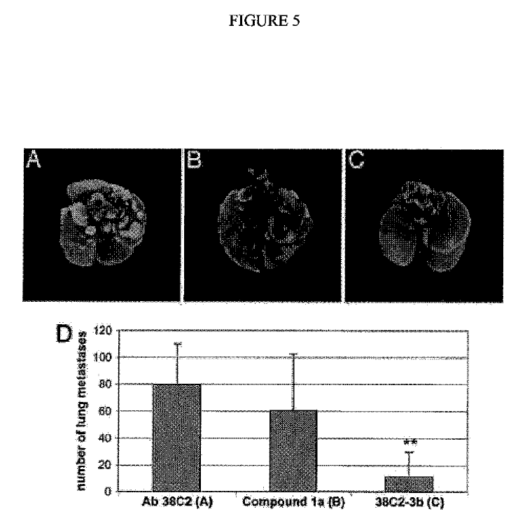

Breast cancer is treatable if diagnosed early. Nevertheless, the prognosis is considerably worse if patients have secondary tumors in distant organs. Prevention of breast cancer metastasis is clearly a significant goal. Both a small-molecule αᵥβ₃ integrin antagonist (22) and an antibody specific for αᵥβ₃ (23) have shown remarkable efficacy in preventing the breast cancer metastasis by interfering with the αᵥβ₃-mediated cell adhesion and proliferation. Furthermore, we have demonstrated effective protection against melanoma lung metastases in animal models of experimental metastasis using another integrin targeting cp38C2 (5). To study the therapeutic potential of our new cp38C2 constructs in experimental breast cancer metastasis, in vivo studies were carried out by using 38C2-3b and 38C2-3c, 1a, and MDA-MB-231 cells in a mouse model. Antibody 38C2 alone served as a negative control. Three groups of six immunodeficient SCID mice were intravenously injected with 5×10⁵ MDA-MB-231 cells pretreated with 38C2-3b (50 μg; 0.67 nmol in 3a), compound 1a (0.67 nmol), or 38C2 (50 μg). On days 2 and 4, animals were injected with identical amounts of the same compounds used on day 1. On day 41, all mice were killed, lungs were removed, and tumor foci at the lung surface were counted. Animals treated with 38C2-3b had significantly fewer metastatic foci than those treated with compound 1a or antibody 38C2 alone. FIG. 5 shows representative examples from the different treatment groups and the number of metastatic foci per group. In an independent study, 38C2-3c was evaluated by using the same protocol. Here again, mice treated with the 38C2-3c had fewer lung metastases than mice treated with 38C2 alone. These results demonstrate a significant enhancement in the therapeutic efficacy of the integrin antagonist provided by linkage in the cpAb format. Indeed, studies in melanoma models indicated that the therapeutic effect of small-molecule integrin antagonists could be enhanced at least 1,000-fold by using the cpAb approach (5).

FIG. 5 shows the effect of 38C2-3b on MDA-MB-231 pulmonary metastasis. SCID mice were injected intravenously with MDA-MB-231 cells pretreated with 50 μg of 3802-3b, 0.3 μg of compound 1a, or 50 μg of 38C2, followed by additional treatments on days 2 and 4. Mice were killed on day 41, representative lungs from the treatment groups were harvested, and metastatic foci were counted in representative sections. Sections of lungs from treatment groups 38C2 (A), 1a (B), and 38C2-3b (C) are shown. The mean number of metastatic foci per group (n=5) with standard deviation are shown in D. **, statistical analysis by the Tukey-Kramer multiple comparison test demonstrated that the difference between the 38C2-3b-treated and the 38C2-treated group was significant (P<0.05). The Student t test also revealed significant differences between the 38C2-3b-treated and 38C2-treated group (P<0.01) and the 38C2-3b treated and 1a-treated group (P<0.05).

### CONCLUSIONS

A new strategy for the self-assembly of cpAbs was explored. The approach described in this article combines the advantages of chemistry and biology to create a unique class of immunotherapeutic molecules that engenders advantages of each discipline. The main advantage of the adapters or programming agents reported here over simpler systems such as diketones is that the antibody catalyzed the formation of its own adapter from a proadapter that itself was much less reactive than a diketone. The relatively inertness of the proadapter may present advantages in cases where the programming agent presents chemical groups that are themselves reactive with diketones. The programming agent and antibody can be injected separately, and the complex will be formed in vivo, or alternatively, the complex can be completed in vitro and delivered as a conventional monotherapeutic. Although the administration of two separate moieties may complicate regulatory approval, the regimen has the advantage that a therapeutic index can be established before the drug is activated. For example, an imaging agent can be attached to the proadapter, allowing the physician to monitor localization of a drug before arming the agent with the effector functions of the antibody molecule. Of course, the complex can also be formed in vitro if such preselectivity is not deemed necessary. Such complexes will circulate for >60 h, giving the adapter greatly extended half-life relative to the small molecule and tunable pharmacokinetics. Half-lives of cpAbs in humans are anticipated to be significantly greater than those observed in mice, as is the case for conventional mAbs. Therapeutic studies in experimental breast cancer metastasis models demonstrate the increase in efficacy that can be provided to a small molecule through coupling with an antibody effector.

### Materials and Methods

### Targeting Agents 1a, 2a, 3a, and 3c

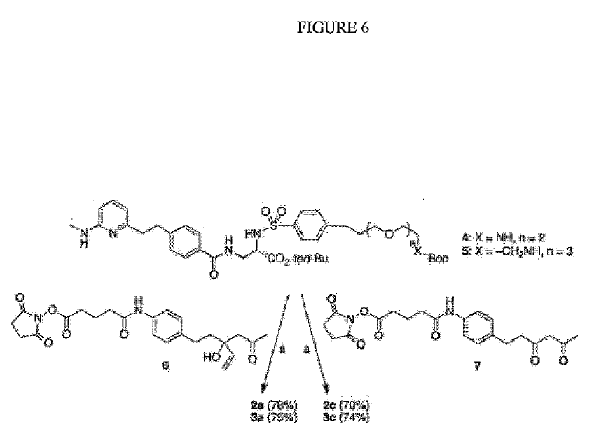

Compound 1a was prepared following the process described for 1 (13). Compound 2c was prepared from its precursor 4 in two steps as described earlier (4). Similarly, compounds 2a, 3a, and 3c were prepared via 4 or 5 and the aldol prolinker 6 or the diketone linker 7, as described below in Scheme 1 (FIG. 6). FIG. 6 shows the synthesis of the αᵥβ₃ integrin-targeting agents; a, trifluoroacetic acid, CH₂Cl₂, anisole, then 6 or 7, Et₃N, and CH₃CN

### Targeting Agent 2a

To a solution of compound 4 (785 mg, 1.0 mmol) in CH₂Cl₂ (3 ml), anisole (1.0 ml) and trifluoroacetic acid (1.0 ml) were added. After 2 h at room temperature, solvents and excess reagents were removed under pressure and taken to the next step without purification. Separately, compound 6 was prepared from the corresponding acid precursor (450 mg; 1.3 mmol), N-hydroxy succinimide (180 mg; 1.56 mmol), 1-(3-dimethylaminopropyl)-3-ethylcarbodiimide hydrochloride (298 mg; 1.56 mmol), and 4-dimethylaminopyridine (8 mg; 0.065 mmol) in CH₂Cl₂ (5 ml) and added to a mixture of the above described deprotection product of 4 and Et₃N (0.5 ml) in CH₃CN (5 ml). After 24 h, solvents were removed under pressure, and the crude mixture was purified by column chromatography over silica gel using CH₂Cl₂-MeOH (9:1) affording pure 2a (745 mg; 78%). R_{f} 0.45 (MeOH—CH₂Cl₂, 1:5); ¹H NMR (500 MHz, CDCl₃): 7.70 (2H, d, J=8.1 Hz), 7.57 (2H, d, J=7.8 Hz), 7.48 (1H, dd, J=7.4, 1.2 Hz), 7.37 (2H, d, J=85 Hz), 7.16 (2H, d, J=8.1 Hz), 7.02-6.99 (4H, m), 6.35 (2H, d, J=8.8 Hz), 6.28 (2H, d, J=7.4 Hz), 5.78 (1H, dd, J=17.2, 10.6 Hz), 5.20 (1H, dd, J=17.2, 1.1 Hz), 5.11 (1H, dd, J=10.6, 1.1 Hz), 3.82 (1H, m), 3.73-3.31 (12H, m), 3.06-3.00 (2H, m), 2.91-2.46 (11H, m), 2.27 (2H, t J=7.4 Hz), 2.18 (2H, J=J 6.6 Hz), 2.09 (3H, s), 1.92-1.88 (2H, m), 1.81-1.63 (4H, m); MS [electrospray ionization (ESI)]: 957 (MH⁺), 979 (MNa⁺); HRMS (ESI-TOF high acc) calculated for C₅₀H₆₅N₆O₁₁S: 957.4353 (MH⁺), found: 957.4348.

### Targeting Agent 3a

This compound (181 mg; 75%) was prepared by deprotection of 5 (200 mg; 0.24 mmol) with trifluoroacetic acid (0.5 ml) in the presence of anisole (0.5 ml) in CH₂Cl₂ (2 ml) followed by reaction of the crude product with the NHS-ester 6 (160 mg; 0.36 mmol) and Et₃N (0.25 ml) in CH₃CN (3.0 ml). R_{f} 0.37 (MeOH—CH₂Cl₂, 1:5); ¹H NMR (500 MHz, CD₃OD+CDCl₃): 7.68 (2H, d, J=8.0 Hz), 7.63 (2H, d, J=8.0 Hz), 7.40-7.36 (3H, m), 7.18-7.14 (4H, m), 7.03 (2H, d, J=8.5 Hz), 6.33 (1H, d, J=7.5 Hz), 6.27 (1H, d, J=8.2 Hz), 5.82 (1H, dd, J=17.5, 11.0 Hz), 5.23 (1H, d, J=17.0 Hz), 5.12 (1H, d, J=11.0 Hz), 3.67 (1H, m), 3.56-3.36 (14H, m), 3.21 (2H, t, J=6.5 Hz), 2.94 (2H, t, J=8.5 Hz), 2.82 (3H, s), 2.84-2.81 (2H, m), 2.76 (1H, m), 2.63-2.47 (5H, m), 2.30 (2H, t, J=7.5 Hz), 2.18 (2H, t, J=7.0 Hz), 2.12 (3H, s), 1.92-1.88 (2H, m), 1.81-1.68 (6H, m); MS (ESI): 1,015 (MH⁺), 1037 (MNa⁺); high-resolution MS (ESI-TOF high acc) calculated for C₅₃H₇₁N₆O₁₂S: 1015.4845 (MH⁺), found-1015.4838.

### Targeting Agent 3c

Compound 3c was prepared (175 mg; 74%) by deprotection of 5 (200 mg; 0.24 mmol) with trifluoroacetic acid (0.5 ml) in the presence of anisole (0.5 ml) in CH₂Cl₂ (2 ml) followed by reaction of the crude product with the NHS-ester 7 (160 mg, 0.36 mmol) and Et₃N (0.25 ml) in CH₃CN (3.0 ml). R_{f} 0.35 (MeOH—CH₂Cl₂, 1:5); ¹H NMR (500 MHz, CD₃OD CDCl₃): 7.74 (2H, d, J=8.0 Hz), 7.67 (2H, d, J=8.0 Hz), 7.49-7.46 (3H, m), 7.23 (2H, d, J=8.0 Hz), 7.18 (2H, d, J=7.5 Hz), 7.11 (2H, d, J=8.0 Hz), 6.38 (1H, d, J=8.0 Hz), 6.35 (1H, d, J=8.5 Hz), 3.63-3.48 (14H, m), 3.43 (2H, t, J=6.0 Hz), 3.28 (2H, t, J=6.2 Hz), 2.99-2.96 (2H, m), 2.91-2.86 (4H, m), 2.89 (3H, s), 2.67 (2H, t, J=7.0 Hz), 2.61 (1H, br, s), 2.56 (2H, t, J=8.0 Hz), 2.35 (2H, t, J=7.5 Hz), 2.24 (2H, t, J=7.5 Hz), 2.04 (3H, s), 1.98-1.92 (2H, m), 1.87-1.81 (2H, m), 1.78-1.73 (2H, m); MS (ESI): 987 (MH⁺), 1,009 (MNa⁺); high-resolution MS (ESI-TOF high acc) calculated for C₅₁H₆₇N₆O₁₂S: 987.4532 (MH⁺), found: 987.4525.

### Antibody, Cell Lines, and Animals

The generation and purification of mouse mAb 38C2 has been described elsewhere. Human breast cancer cell lines MDA-MB-231 and MDA-MB-435 were obtained from the American Type Culture Collection. The MDA-MB-231 cell line was cultured in Leibovitz L-15 medium supplemented with 2 mM L-glutamine and 10% FCS in CO₂-free environment. MDA-MB-435 cells were also supplemented with 0.01 mg/ml insulin. Six-week-old female CB17-SCID mice were purchased from Taconic Farms, and eight-week-old BALB c mice were obtained from the in-house animal facility. Anti-integrin αᵥβ₃ antibody LM609 (1 mg/ml), anti-αᵥβ₅ antibody P1F6 (1 mg/ml), and FITC conjugated goat anti-mouse antibody (catalog no. AP124F; 1 mg/ml) were purchased from Chemicon International (Temecula, Calif.), and Immuno-Fluore mounting medium (catalog no. 62270) was from MP Biomedicals (Aurora, Ohio).

Formation of Antibody Construct and Evaluation of Binding to αᵥβ₃ Integrin-Expressing Cells

The generation and purification of mouse mAb 38C2 has been described elsewhere. The antibody constructs (38C2-2b, -2c, -3b, and -3c) were prepared by mixing a solution of compound 2a, 2c, 3a, or 3c (100 μM; 3.3 μl) with antibody 38C2 (50 M; 3.3 μl) in PBS buffer (total volume, 50 μl), and the mixtures were incubated at 37° C. for 2 h. Cells were detached by brief trypsinization with 0.25% (wt/vol) trypsin, 1 mM EDTA, washed with PBS, and resuspended at a concentration of 10⁶ cells per milliliter in flow cytometry buffer [1% (wt/vol) BSA 25 mM Hepes in PBS, pH 7.4]. Aliquots of 100 μl containing 10⁵ cells were distributed into wells of a V-bottom 96-well plate (Corning) for indirect immunofluorescence staining in the presence of serial dilutions (1:20, 1:100, 1:500, and 1:2500) of cpAbs 38C2-2b, 38C2-2c, 38C2-3b, or 38C2-3c in flow cytometry buffer. After the constructs were incubated with cells for 1 h, FITC-conjugated goat anti-mouse polyclonal antibodies (a 1:100 dilution; i.e., 10 μg/ml, in flow cytometry buffer) were added to the mixture and further incubated for 45 min at room temperature. Finally, samples were analyzed by flow cytometry using a FACScan instrument (Becton Dickinson).

For the in vivo antibody construct formation, three 8-week-old BALB c mice were injected i.v. (tail vein) with 100 μl of 10 mg/ml antibody 38C2 in PBS Compounds 2a and 2c were injected i.p. as 100 μl of 10 mg/ml in 50% PBS/25% DMSO/25% ethanol. Sera were prepared by centrifuging eye bleeds taken 24, 48, 72, 96, and 168 h after the injections. By using a 1:100 dilution in flow cytometry buffer, the prepared sera were analyzed by flow cytometry as described above.

### Cell-Uptake of the Antibody Constructs

Cover slides in 24-well plates were kept under UV for 2 h. MDA-MB-231 cells were detached by brief trypsinization with 0.25% (wt/vol) trypsin/1 mM EDTA, washed with PBS, and resuspended at a concentration of 1.5×10⁶ cells per milliliter. Aliquots of 100 μl containing 150,000 cells were distributed into wells. The cells were incubated with 38C2-3b or 382-3c (5 μg/ml, prepared as before using 1 eq of 38C2 and 2 eq of 3a or 3c, respectively) at 37° C. for 15 min and then fixed using 2% paraformaldehyde in 0.01 M PBS for 10 min followed by 0.2% Triton X-100 in PBS at room temperature for 2 min. After the cells were rinsed with PBS containing 0.001% Triton X-100, they were incubated with 10% normal goat serum at room temperature for 60 min and again rinsed with PBS containing 0.001% Triton X-100. Cells were next incubated with FITC-conjugated goat anti-mouse at room temperature for 1 h, rinsed using PBS containing 0.001% Triton X-100, incubated with DAPI (1:500 dilution; i.e., 10 μg ml) (Molecular Probes) at room temperature for 60 min, and mounted onto slides using the Immuno-Fluore mounting medium. Fixed and stained samples were then viewed by using a Rainbow Radiance 2100 laser scanning confocal system attached to a Nikon TE200-U inverted microscope (Bio-Rad). Images were acquired using LASER SHARP 2000 (Bio-Rad) software and processed in LSM EXAMINER (Zeiss) software.

### Prevention of Breast Cancer Metastasis

MDA-MB-231 cells (1×10⁶) were suspended in 100 μl of serum-free medium and injected into the tail vein in 6-week-old female CB17-SCID mice, including 38C2-3b (50 μg; 0.67 nmol in 3a), compound 1a (0.67 nmol), 38C2 (50 μg), and buffer alone. Animals were further injected on days 2 and 4, with the identical amounts of the construct, compound, or antibody. On day 41, all mice were killed, lungs were removed, and tumor foci at the lung surface were counted by anatomy microscope. Statistical analysis by the Tukey-Kramer multiple comparison test demonstrated a significant difference between the 38C2-3b-treated and 38C2-treated group (P<0.05). Student's t test also revealed significant differences between the 38C2-3b-treated and 38C2-treated group (P<0.01) and the 38C2-3b-treated and 1a-treated group (P<0.05). All of the animal experiments were approved by the Institutional Animal Care and Use Committee of the Scripps Research Institute before the experiments were started.

### REFERENCES

The following references are specifically applicable to the Example and are incorporated herein by reference; these references are referenced in the Example by the reference numbers assigned to them.
1. O'Mahony, D. & Bishop, M. R. (2006) Front. Biosci. 11:1620-1635.
2. Chester, K., Pedley, B., Tolner, B., Violet, J., Mayer, A., Sharma, S., Boxer, G., Green, A., Nagl, S. & Begent, R. (2004) Tumor Biol. 25: 91-98.
3. Rader, C., Sinha, S. C., Popkov, M., Lerner, R. A. & Barbas, C. F., III (2003) Proc. Natl. Acad. Sci. USA 100: 5396-5400.
4. Li, L. S., Rader, C., Matsushita, M., Das, S., Barbas, C. F., III, Lerner, R. A. & Sinha, S. C (2004) J. Med. Chem. 47: 5630-5640.
5. Popkov, M., Rader, C., Gonzalez, B., Sinha, S. C. & Barbas, C. F., III (Mar. 28, 2006) Int. J. Cancer: 10.1002 ijc.21924.
6. Wagner, J., Lerner, R. A. & Barbas, C. F., III (1995) Science 270: 17971800.
7. Barbas, C. F., III, Heine, A., Zhong, G., Hoffmann, T., Gramatikova, S., Bjornestedt, R., List, B., Anderson, J., Stura, E. A., Wilson, E. A. & Lerner, R. A. (1997) Science 278: 2085-2092.
8. Mousa, S. A. (2000) Emerg. Ther. Targets 4: 143-153.
9. Liapis, H., Flath, A. & Kitazawa, S. (1996) Diagn. Mol. Pathol. 5:127-135.
10. Stupack, D. G. & Cheresh, D. A. (2004) Curr. Top. Dev. Biol. 64: 207-238.
11. Rader, C., Turner, J. M, Heine, A., Shabat, D., Sinha, C. C, Wilson, I. A, Lerner, R. A & Barbas, C. F., III (2003) J. Mol. Biol. 332: 889-899.
12. List, B., Shabat, D., Zhong, G., Turner, J. M., Li, A., Bui, T., Anderson, J., Lerner, R. A. & Barbas, C. F., III (1999) J. Am. Chem. Soc. 121: 7283-7291.
13. Duggan, M. E., Duong, L. T., Fisher, J. E., Hamill, T. G., Hoffman, W. F., Huff, J. R., Ihie, N. C., Leu, C.-T., Nagy, R, M., Perkins, J. J., et al. (2000) J. Med. Chem. 43: 3736-3745.
14. List, B., Barbas, C. F., III & Lerner, R. A. (1998) Proc. Natl. Acad. Sci. USA 95:15351-15355.
15. Meyer, T., Marshall, J. F. & Hart, I. R. (1998) Br. J. Cancer 77: 530-536.

16. Felding-Habermann, B., O'Toole, T. E., Smith, J. W., Fransvea, E., Ruggeri, Z. M., Ginsberg, M. H., Hughes, P. E., Pampori, N., Shattil, S. J., Saveni, A. & Mueller, B. M. (2001) Proc. Natl. Acad. Sci. USA 98: 1853-1858.
17. Shabat, D., Rader, C., List, B., Lerner, R. A. & Barbas, C. F., III (1999) Proc. Natl. Acad, Sci. USA 96: 6925-6930.
18. Shabat, D., Lode, H. N., Perti, U., Reisfeld, R. A., Rader, C., Lerner, R. A. & Barbas, C. F., III (2001) Proc. Natl. Acad. Sci. USA 98: 7528-7533.
19. Wickham, T. J., Mathias, P., Cheresh, D. A. & Nemerow, G. R. (1993) Cell 73: 309-319.
20. Castel, S., Pagan, R., Mitjans, F., Piulats, J., Goodman, S., Jonczyk, A., Huber, F., Vilaro, S. & Reina, M. (2001) Lab. Invest. 81: 1615-1626.
21. Cullander, C (1999) Methods Mol. Biol. 122: 59-73.
22. Shannon, K. E., Keene, J. L., Settle, S. L., Westlin, T. D., Schroeter, S., Ruminski, P. G. & Griggs, D. W. (2004) Clin. Exn. Metastasis 21: 129-138.
23. Felding-Habermann, B., Lerner, R. A., Lillo, A., Zhuang, S., Weber, M. R., Arrues, S., Gao, C., Mao, S., Saven, A. & Janda, K. D. (2004) Proc. Natl. Acad. Sci. USA 101: 17210-17215.

### EXAMPLE 2

An additional proadapter (formula (V)) incorporating a tertiary aldol moiety was synthesized together with its diketone analogue (formula (VI).

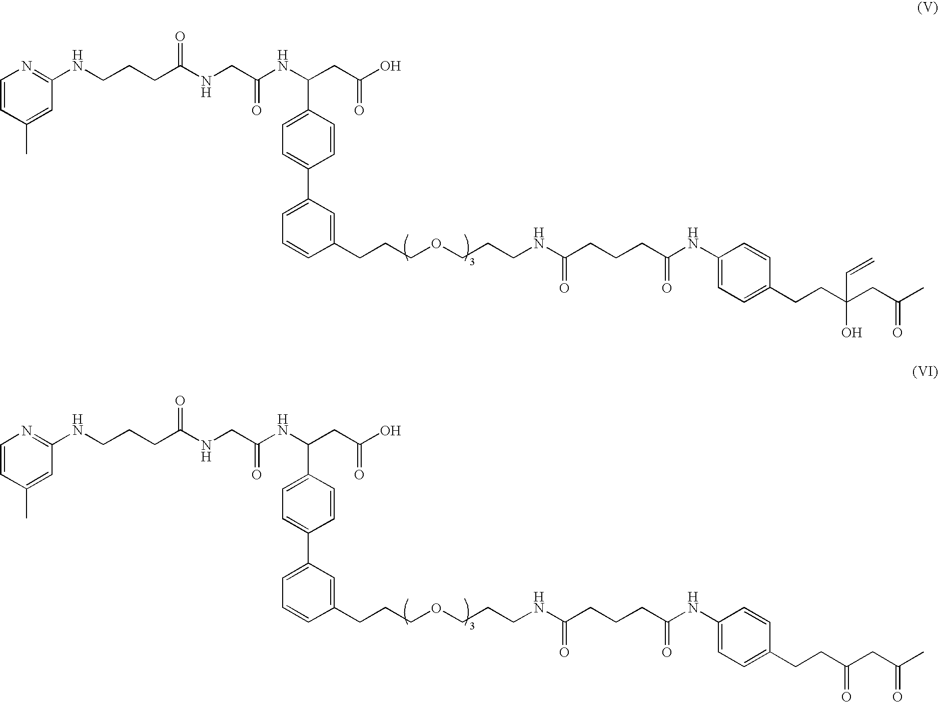

Compounds of formula (V) and formula (VI) have been formulated to target integrin αᵥβ₆ that is expressed on several cancer cell lines. These compounds are based on the compounds described in PCT Patent Publication No. WO 00/48996 by Hölzemann et al. and U.S. Pat. No. 6,576,637 to Hölzemann et al., both incorporated by this reference, as well as in S. L. Goodman et al., “Nanomolecular Small Molecular Inhibitors for αvβ₆, αvβ₅, and αvβ₃ Integrins,” J. Med. Chem. 45: 1045-1051 (2000), all of which are incorporated by this reference.

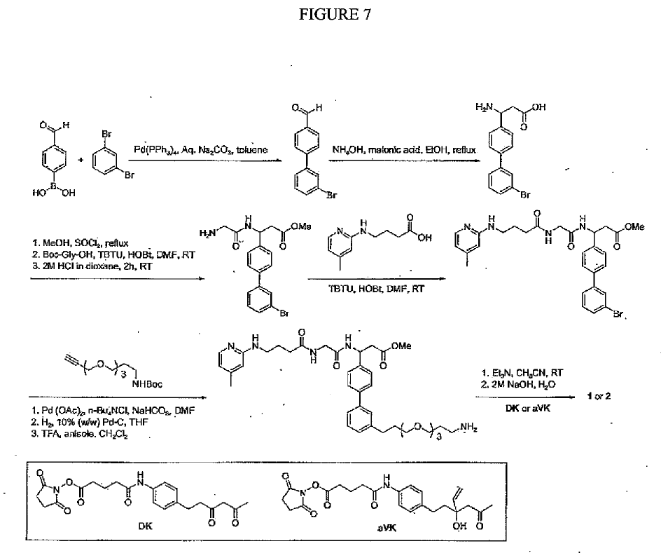

The reaction scheme is shown in Scheme 2 (FIG. 7). The reactant TBTU in Scheme 2 is O-(benzotriazol-1-yl)-N,N,N′,N′-tetramethyluronium tetrafluoroborate. The reactant HOBt is N-hydroxybenzotriazole.

In the reactions shown in Scheme 2 (FIG. 7), the compounds of Formula (IV) and (V) were prepared starting with 4-formylphenylboronic acid and 1,3-dibromobenzene as shown in Scheme 2. Thus, the 4-formylphenylboronic acid underwent Suzuki reaction with 1,3-dibromobenzene, and the resulting aldehyde was reacted with ammonium hydroxide and malonic acid affording a β-amino acid. The latter was esterified as methyl ester and reacted with Boc-protected-glycine giving the corresponding dipeptide, which underwent Boc-deprotection followed by a peptide coupling with 4-amino-2′-(4′-methyl)pyridylbutyric acid. The resulting bromobiphenyl tripeptide was coupled with an alkyne linker. The product was hydrogenated and deprotected giving a free amino acid, which reacted with the NHS ester of a diketone acid (DK) or an acetone adduct of the vinyl ketone (aVK) linker affording compounds of Formula (VI) and Formula (V), respectively.

### ADVANTAGES OF THE INVENTION

The present invention provides a powerful and versatile method for the generation of chemically programmed antibodies that allows the construction of molecules that have different specificities using the same antibody. This breaks the “one antibody-one-target” axiom and greatly extends the range of targeted antibody therapy by enabling the formation of a wide variety of targeted antibodies without the need of generating individual antibodies specific for each antigen. The present invention also makes use of the effector functions of intact antibody molecules, particularly those mediated by the Fc portion of the molecule. The use of the catalytic activity of the antibody itself to activate a reactant necessary for labeling provides great specificity and avoids the use of reagents that can react with multiple potentially reactive residues, such as lysine residues, in a protein molecule. This reaction unveils a reactive vinyl ketone without the necessity of introducing such a reactive reagent directly to the protein. This “unveiling” approach” reduces cross-reactivity and allows more efficient purification of the desired product; it also reduces the possible introduction of immunogenic groups that could conceivably result in antibody generation when the antibody molecules are used in vivo.

Methods and compositions according to the present invention have a large number of possible uses because of the wide variety of signal modules that could be coupled to the antibody in this “unveiling” reaction. However, methods and compositions according to the present invention are promising for the prevention of metastasis, particularly in breast cancer and melanoma. The methods are flexible and have broad application, allowing labeling with a variety of linkers or without a linker, and allow the incorporation of labeled molecules into larger fusion proteins.

The present invention also provides for the use of chemically programmed antibodies in diagnosis and treatment. Chemically programmed antibodies according to the present invention can be used either in vitro or in vivo in a large number of diagnostic procedures, including immunostaining and immunolabeling. Labeled cells can be sorted, detected, and quantitated using fluorescence-activated cell sorting (FACS) or other techniques. Chemically programmed antibodies according to the present invention can also be used in methods of treatment and can be formulated into pharmaceutical compositions.

Compositions and methods according to the present invention possess industrial applicability for the preparation of medicaments to treat a number of diseases and conditions, including cancer. They also possess industrial applicability in their use for diagnosis.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

We claim:

1. A method for producing a chemically programmed antibody comprising the steps of:
(a) reacting a conjugate comprising a signal module covalently linked to a proadapter with a catalytic moiety selected from the group consisting of a catalytic antibody and a Fab fragment of a catalytic antibody, wherein the proadapter includes therein a precursor to a reactive moiety activated to a reactive moiety by a reaction catalyzed by the catalytic moiety; and
(b) crosslinking the reactive moiety to a side chain of an amino acid residue in the active site of the catalytic moiety to produce the chemically programmed antibody.

2. The method of claim 1 wherein the reaction catalyzed by the catalytic moiety is a retro-aldol reaction.

3. The method of claim 2 wherein the catalytic moiety has aldolase activity.

4. The method of claim 1 wherein the catalytic moiety is a catalytic antibody.

5. The method of claim 1 wherein the catalytic moiety is a Fab fragment of a catalytic antibody.

6. The method of claim 4 wherein the catalytic antibody is antibody 38C2.

7. The method of claim 5 wherein the Fab fragment of the catalytic antibody is a Fab fragment of antibody 38C2.

8. The method of claim 2 wherein the proadapter includes therein a tertiary aldol moiety.

9. The method of claim 8 wherein the tertiary aldol moiety is converted to a reactive vinyl ketone moiety by the retro-aldol reaction catalyzed by the catalytic moiety.

10. The method of claim 9 wherein the vinyl ketone moiety reacts with a lysine residue in the active site of the catalytic moiety via Michael addition.

11. The method of claim 1 wherein the proadapter comprises a molecule selected from the group consisting of: 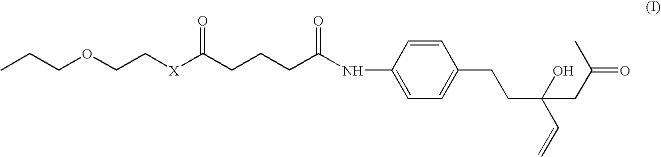
(a) a molecule of formula (I) wherein X is selected from the group consisting of NH and —CH₂NH and n is 2 or 3; and
(b) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule.

12. The method of claim 11 wherein the proadapter comprises a molecule of formula (I) wherein X is NH and n is 2.

13. The method of claim 11 wherein the proadapter comprises a molecule of formula (I) wherein X is —CH₂NH and n is 3.

14. The method of claim 1 wherein the proadapter comprises a molecule selected from the group consisting of: 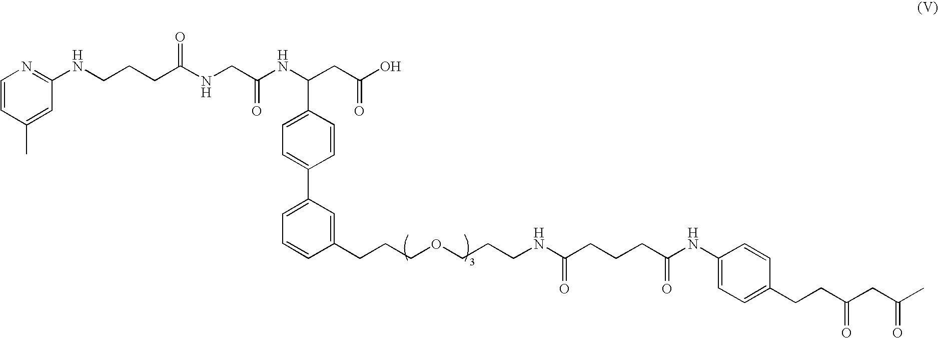
(a) a molecule of formula (V); and
(b) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule.

15. The method of claim 14 wherein the proadapter comprises a molecule of formula (V).

16. The method of claim 1 wherein the signal module specifically targets an integrin.

17. The method of claim 14 wherein the integrin is selected from the group consisting of αᵥβ₃, αᵥβ₅, and αᵥβ₆.

18. The method of claim 17 wherein the integrin is αᵥβ₃.

19. The method of claim 17 wherein the integrin is αᵥβ₅.

20. The method of claim 17 wherein the integrin is αᵥβ₆.

21. The method of claim 20 wherein the proadapter is selected from the group consisting of:
(a) a molecule of formula (V); and
(b) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule.

22. The method of claim 21 wherein the proadapter is a molecule of formula (V).

23. The method of claim 1 wherein the signal module comprises an RGD peptidomimetic.

24. The method of claim 1 wherein the signal module is a modified T-20 peptide having the amino acid sequence N-Acetyl-YTSLIHSLIEESQNQQEKNEQELLELDKWASLWNWFC (SEQ ID NO: 1).

25. The method of claim 1 wherein the signal module is a nilutamide analog that targets the androgen receptor.

26. The method of claim 1 wherein the signal module is a fluorescent, chemiluminescent, or bioluminescent molecule or a molecule incorporating a detectable radioisotope.

27. The method of claim 1 wherein the signal module is a protein.

28. The method of claim 27 wherein the signal module is a protein that is an enzyme that catalyzes a reaction that produces a detectable product.

29. The method of claim 27 wherein the signal module is a protein that is detected by the use of a secondary labeled antibody that specifically binds the signal module.

30. The method of claim 27 wherein the protein is a receptor.

31. The method of claim 30 wherein the receptor is a thrombospondin receptor.

32. The method of claim 1 wherein the method is performed in vitro.

33. The method of claim 1 wherein the method is performed in vivo.

34. A chemically programmed antibody formed by the method of claim 1.

35. The chemically programmed antibody of claim 34 wherein the antibody incorporates a 38C2 catalytic antibody.

36. The chemically programmed antibody of claim 34 wherein the antibody incorporates a Fab fragment of 38C2 catalytic antibody.

37. The chemically programmed antibody of claim 34 wherein the proadapter comprises a molecule selected from the group consisting of: 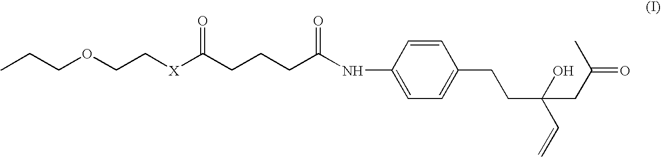
(a) a molecule of formula (I) wherein X is selected from the group consisting of NH and —CH₂NH and n is 2 or 3; and
(b) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule.

38. The chemically programmed antibody of claim 37 wherein the proadapter comprises a molecule of formula (I) wherein X is NH and n is 2.

39. The chemically programmed antibody of claim 37 wherein the proadapter comprises a molecule of formula (I) wherein X is —CH₂NH and n is 3.

40. The chemically programmed antibody of claim 34 wherein the proadapter comprises a molecule selected from the group consisting of: 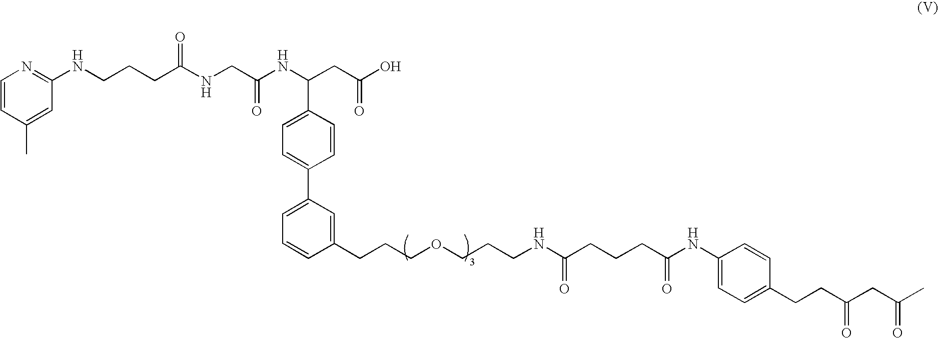
(a) a molecule of formula (V); and
(b) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule.

41. The chemically programmed antibody of claim 40 wherein the proadapter comprises a molecule of formula (V).

42. The chemically programmed antibody of claim 34 wherein the signal module specifically targets an integrin.

43. The chemically programmed antibody of claim 42 wherein the integrin is selected from the group consisting of αᵥβ₃, αᵥβ₅, and αᵥβ₆.

44. The chemically programmed antibody of claim 43 wherein the integrin is αᵥβ₃.

45. The chemically programmed antibody of claim 43 wherein the integrin is αᵥβ₅.

46. The chemically programmed antibody of claim 43 wherein the integrin is αᵥβ₆.

47. The chemically programmed antibody of claim 46 wherein the proadapter is selected from the group consisting of:
(a) a molecule of formula (V); and
(b) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule.

48. The chemically programmed antibody of claim 47 wherein the proadapter is a molecule of formula (V).

49. The chemically programmed antibody of claim 34 wherein the signal module comprises an RGD peptidomimetic.

50. The chemically programmed antibody of claim 34 wherein the signal module is a modified T-20 peptide having the amino acid sequence N-Acetyl-YTSLIHSLIEESQNQQEKNEQELLELDKWASLWNWFC (SEQ ID NO: 1).

51. The chemically programmed antibody of claim 34 wherein the signal module is a nilutamide analog that targets the androgen receptor.

52. The chemically programmed antibody of claim 34 wherein the signal module is a fluorescent, chemiluminescent, or bioluminescent molecule or a molecule incorporating a detectable radioisotope.

53. The chemically programmed antibody of claim 34 wherein the signal module is a protein.

54. The chemically programmed antibody of claim 53 wherein the signal module is a protein that is an enzyme that catalyzes a reaction that produces a detectable product.

55. The chemically programmed antibody of claim 53 wherein the signal module is a protein that is detected by the use of a secondary labeled antibody that specifically binds the signal module.

56. The chemically programmed antibody of claim 53 wherein the protein is a receptor.

57. The chemically programmed antibody of claim 56 wherein the receptor is a thrombospondin receptor.

58. The chemically programmed antibody of claim 42 wherein the antibody is internalized in cells via an integrin-dependent endocytosis pathway.

59. A method of treatment of a disease or condition treatable by the administration of a chemically programmed antibody comprising the step of administering a therapeutically effective quantity of the chemically programmed antibody of claim 34 to treat the disease or condition.

60. The method of claim 59 wherein the chemically programmed antibody is administered in a pharmaceutical composition.

61. The method of claim 59 wherein the disease or condition is cancer.

62. The method of claim 61 wherein the cancer is metastatic breast cancer, and the treatment results in the prevention of metastasis.

63. The method of claim 61 wherein the cancer is metastatic melanoma, and the treatment results in the prevention of metastasis.

64. A method of treatment of a disease or condition treatable by the administration of a chemically programmed antibody comprising the steps of:
(a) administering a therapeutically effective quantity of a catalytic moiety that can form the chemically programmed antibody of claim 1 when reacted with a proadaptor;
(b) administering a therapeutically effective quantity of a proadaptor that can form the chemically programmed antibody of claim 1 when reacted with the catalytic moiety; and
(c) reacting the catalytic moiety and proadaptor in vivo to form the chemically programmed antibody in a therapeutically effective quantity to treat the disease or condition.

65. The method of claim 64 wherein the catalytic moiety is administered in a pharmaceutical composition.

66. The method of claim 64 wherein the proadaptor is administered in a pharmaceutical composition.

67. The method of claim 64 wherein the catalytic moiety and the proadaptor are administered in a single pharmaceutical composition.

68. The method of claim 64 wherein the disease or condition is cancer.

69. The method of claim 68 wherein the cancer is metastatic breast cancer, and the treatment results in the prevention of metastasis.

70. The method of claim 68 wherein the cancer is metastatic melanoma, and the treatment results in the prevention of metastasis.

71. A method of treatment of a disease or condition treatable by the administration of a chemically programmed antibody comprising the steps of:
(a) administering a therapeutically effective quantity of a nucleic acid encoding a catalytic moiety that can form the chemically programmed antibody of claim 1 when reacted with a proadaptor;
(b) administering a therapeutically effective quantity of a proadaptor that can form the chemically programmed antibody of claim 1 when reacted with the catalytic moiety; and
(c) reacting the catalytic moiety and proadaptor in vivo to form the chemically programmed antibody in a therapeutically effective quantity to treat the disease or condition.

72. The method of claim 71 wherein the nucleic acid is administered in a pharmaceutical composition.

73. The method of claim 71 wherein the proadaptor is administered in a pharmaceutical composition.

74. The method of claim 71 wherein the nucleic acid and the proadaptor are administered in a single pharmaceutical composition.

75. The method of claim 71 wherein the disease or condition is cancer.

76. The method of claim 75 wherein the cancer is metastatic breast cancer, and the treatment results in the prevention of metastasis.

77. The method of claim 75 wherein the cancer is metastatic melanoma, and the treatment results in the prevention of metastasis.

78. A proadapter comprising a molecule selected from the group consisting of: 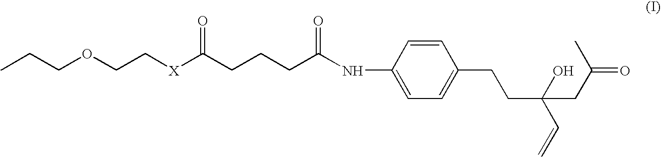
(a) a molecule of formula (I) wherein X is selected from the group consisting of NH and —CH₂NH and n is 2 or 3; and
(b) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule.

79. The proadapter of claim 78 wherein the proadapter comprises a molecule of formula (I) wherein X is NH and n is 2.

80. The proadapter of claim 78 wherein the proadapter comprises a molecule of formula (I) wherein X is —CH₂NH and n is 3.

81. A proadapter comprising a molecule selected from the group consisting of: 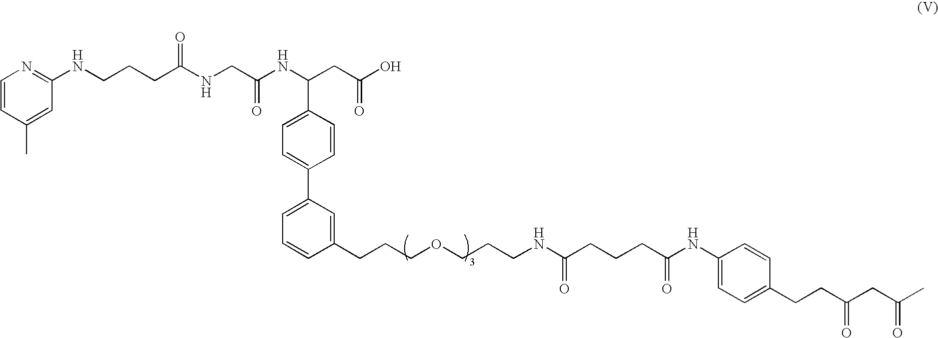
(a) a molecule of formula (V); and
(b) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule.

82. The proadapter of claim 81 wherein the proadapter is a molecule of formula (V).

83. A conjugate comprising:
(a) a proadapter comprising a molecule selected from the group consisting of: 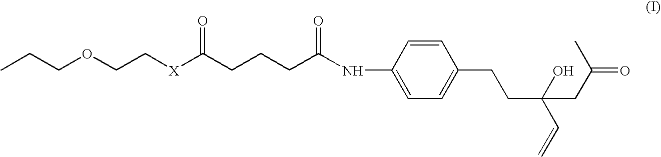
(i) a molecule of formula (I) wherein X is selected from the group consisting of NH and —CH₂NH and n is 2 or 3; and
(ii) a derivative of a molecule of formula (I) wherein X is selected from the group consisting of NH and —CH₂NH and n is 2 or 3 in which the molecule is substituted with one to five substituents each independently selected from the group consisting of lower alkyl, hydroxyl, alkoxy, and halo and such that the derivative substantially retains the activity with the catalytic moiety of the underivatized molecule; and
(b) a signal module covalently linked to the proadapter.

84. The conjugate of claim 83 wherein the proadapter comprises a molecule of formula (I) wherein X is NH and n is 2.

85. The conjugate of claim 83 wherein the proadapter comprises a molecule of formula (I) wherein X is —CH₂NH and n is 3.

86. The conjugate of claim 83 wherein the signal module specifically targets an integrin.

87. The conjugate of claim 86 wherein the integrin is selected from the group consisting of αᵥβ₃ and αᵥβ₅.

88. The conjugate of claim 87 wherein the integrin is αᵥβ₃.

89. The conjugate of claim 87 wherein the integrin is αᵥβ₅.

90. The conjugate of claim 83 wherein the signal module comprises an RGD peptidomimetic.

91. The conjugate of claim 83 wherein the signal module is a modified T-20 peptide having the amino acid sequence N-Acetyl-YTSLIHSLIEESQNQQEKNEQELLELDKWASLWNWFC (SEQ ID NO: 1).

92. The conjugate of claim 83 wherein the signal module is a fluorescent, chemiluminescent, or bioluminescent molecule or a molecule incorporating a detectable radioisotope.

93. The conjugate of claim 83 wherein the signal module is a protein.

94. The conjugate of claim 93 wherein the signal module is a protein that is an enzyme that catalyzes a reaction that produces a detectable product.

95. The conjugate of claim 93 wherein the signal module is a protein that is detected by the use of a secondary labeled antibody that specifically binds the signal module.

96. The conjugate of claim 93 wherein the protein is a receptor.

97. The conjugate of claim 96 wherein the receptor is a thrombospondin receptor.

98. The conjugate of claim 83 wherein the signal module is a nilutamide analog that targets the androgen receptor.

99. A conjugate comprising:
(a) a proadapter comprising a molecule selected from the group consisting of: 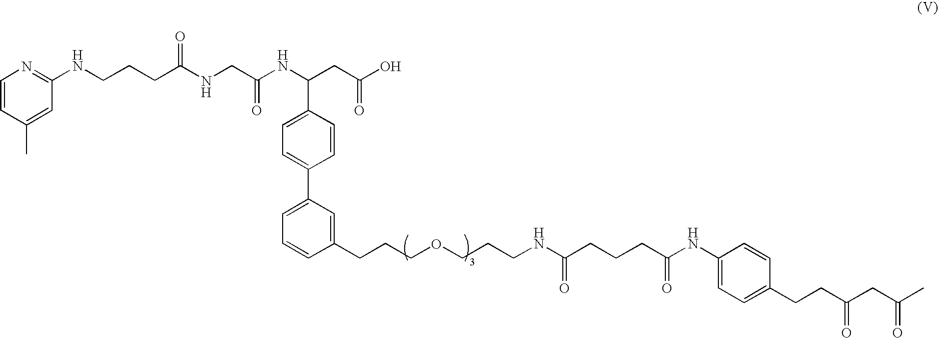
(i) a molecule of formula (V); and
(ii) a derivative of a molecule of formula (V) in which the molecule is substituted with one to five substituents each independently selected from the group consisting of lower alkyl, hydroxyl, alkoxy, and halo and such that the derivative substantially retains the activity with the catalytic moiety of the underivatized molecule; and
(b) a signal module covalently linked to the proadapter.

100. The conjugate of claim 99 wherein the proadapter is a molecule of formula (V).

101. The conjugate of claim 99 wherein the signal module specifically targets an integrin.

102. The conjugate of claim 101 wherein the integrin is αᵥβ₆.

103. The conjugate of claim 99 wherein the signal module comprises an RGD peptidomimetic.

104. The conjugate of claim 99 wherein the signal module is a modified T-20 peptide having the amino acid sequence N-Acetyl-YTSLIHSLIEESQNQQEKNEQELLELDKWASLWNWFC (SEQ ID NO: 1).

105. The conjugate of claim 99 wherein the signal module is a fluorescent, chemiluminescent, or bioluminescent molecule or a molecule incorporating a detectable radioisotope.

106. The conjugate of claim 99 wherein the signal module is a protein.

107. The conjugate of claim 106 wherein the signal module is a protein that is an enzyme that catalyzes a reaction that produces a detectable product.

108. The conjugate of claim 106 wherein the signal module is a protein that is detected by the use of a secondary labeled antibody that specifically binds the signal module.

109. The conjugate of claim 106 wherein the protein is a receptor.

110. The conjugate of claim 109 wherein the receptor is a thrombospondin receptor.

111. The conjugate of claim 99 wherein the signal module is a nilutamide analog that targets the androgen receptor.
